SEQUENCE LISTING (22 sequences)

SEQ ID NO: 1 - protein, 37 aa, Artificial Sequence
  Synthetic Polypeptide
ACDTATCVTHRLAGLLSRSGGVVKNNFVPTNVGSKAF

SEQ ID NO: 2 - protein, 13 aa, Artificial Sequence
  Synthetic Polypeptide
MSAWSHPQFEKGA

SEQ ID NO: 3 - protein, 11 aa, Artificial Sequence
  Synthetic Polypeptide
GGSGGPLGIAG

SEQ ID NO: 4 - protein, 8 aa, Artificial Sequence
  Synthetic Polypeptide
GGSGDEVD

SEQ ID NO: 5 - protein, 8 aa, Artificial Sequence
  Synthetic Polypeptide
GGSGIEGR

SEQ ID NO: 6 - protein, 11 aa, Artificial Sequence
  Synthetic Polypeptide
GGSGENLYFQG

SEQ ID NO: 7 - protein, 9 aa, Artificial Sequence
  Synthetic Polypeptide
GGSGDDDDK

SEQ ID NO: 8 - protein, 7 aa, Artificial Sequence
  Synthetic Polypeptide
GPLGIAG

SEQ ID NO: 9 - protein, 4 aa, Artificial Sequence
  Synthetic Polypeptide
DEVD

SEQ ID NO: 10 - protein, 4 aa, Artificial Sequence
  Synthetic Polypeptide
IEGR

SEQ ID NO: 11 - protein, 7 aa, Artificial Sequence
  Synthetic Polypeptide
ENLYFQG

SEQ ID NO: 12 - protein, 5 aa, Artificial Sequence
  Synthetic Polypeptide
DDDDK

SEQ ID NO: 13 - protein, 4 aa, Artificial Sequence
  Synthetic Polypeptide
GGSG

SEQ ID NO: 14 - protein, 251 aa, Artificial Sequence
  StrepII-GFP
MSAWSHPQFEKGAVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 15 - protein, 38 aa, Artificial Sequence
  haCGRP-Gly
ACDTATCVTHRLAGLLSRSGGVVKNNFVPTNVGSKAFG

SEQ ID NO: 16 - DNA, 3154 bp, Artificial Sequence
  pEXPR-T7-cfSGFP2-Linker-Cleavage-Site-CGRP-Gly
gcttgtgacacagccacctgtgtcacccaccgcctggccggtctgctgtctcgctcgggtggcgttgttaaaaacaattttgtgccaaccaatgttggttcaaaagcgtttggctaacaaagcccgaaaggaagctgagttggctgctgccaccgctgagcaataactagcataaccccttggggcctctaaacgggtcttgaggggttttttgctgaaaggaggaactatatccggatatccacaggacggaggtagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctggtgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgctagcatggatctcggggacgtctaactactaagcgagagtagggaactgccaggcatcaaataaaacgaaaggcccagtcttccgactgagcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcagaggatcgagatctcgatcccgcgaaattaatacgactcactatagggagagccacaacggtttccctctagaaataattttgtttaactttaagaaggagatatacatatgagcgcgtggagccatccgcagtttgaaaaaggtgctgtatccaagggcgaagagctgttcacgggcgtagtaccgattttggtggagctggacggcgatgtgaacggccataaatttagtgtgagcggcgaaggtgagggcgatgcgacatatggcaaactgaccctgaaatttattagtaccaccggtaaactgccagtgccttggccgacgctggttaccaccctgacctacggtgtgcaaatgttcgctcgctaccccgaccatatgaaacagcatgacttcttcaagagcgcaatgccggaaggttacgtgcaggagcgtacgatttttttcaaagatgatgggaattacaagacacgtgcggaagtgaaattcgagggcgatacgctggttaatcgcatcgaactgaaaggcatcgacttcaaagaagatggtaacattctgggtcacaagcttgaatacaactacaactcacataacgtatatatcacagcagataagcagaagaatggtattaaggcgaatttcaagatccgtcacaatattgaggatggcggtgttcagttagctgatcattatcaacagaacacccctattggagatggcccagtgttgctgccagataatcattacctgtctacccagagcaaactgagcaaagaccctaatgagaaacgtgatcatatggtcttactggagttcgttacagcggcgggcattacgctgggtatggatgagctgtataagggtggctcaggtgatgaagtagat

SEQ ID NO: 17 - DNA, 12014 bp, Artificial Sequence
  pLV-hEF1a-Glo22F-IRES-H2B-Cerulean-2A-puro
ctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagcctggccaccatgcctggcgcagtaggcaaggtggtgcccttcatcgaggctaaggtggtggacttggacactggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttagtggacgaggtgcctaaaggactgaccggcaagttggacgcccgcaagatccgcgagattctcattaaggccaagaagggatccaattgggattctgggtgctccagagaaggtatgtatgaaagctttattgagtcactgccattccttaaatctttggagttttctgcacgcctgaaagtagtagatgtgataggcaccaaagtatacaacgatggagaacaaatcattgctcagggagattcggctgattcttttttcattattgaatctggagaagtgaaaattactatgaaaagaaagggtaaatcagaagtggaagagaatggtgcagtagaaatcgctcgatgctcgcggggacagtactttggagagcttgccctggtaactaacaaacctcgagcagcttctgcccacgccattgggactgtcaaatgtttagcaatggatgtgcaagcatttgaaaggcttctgggaccttgcatggaaattatgaaaaggaacatcgctacctatgaagaacagttagttgccctgtatggaacgaacatggatattgtagccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcactccagaaggggtttaagactgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacagccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaaggagggccgcggcagcctgctgacctgcggcgacgtggaggaaaaccccggccccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtgtcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagggacaggtgatatccagcacagtggcggccgctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattctgcagatatccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccccctgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttaaatggtacctttaagaccaatgacttacaaggcagctgtagatcttagccactttttaaaagaaaaggggggactggaagggctagctcactcccaacgaagacaagatctgctttttgcttgtactgggtctctctggttagaccagatctgagcctgggagctctctggctgcctagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgacgtgtgtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttattccagtgtggtggaattctgcagtctgc

SEQ ID NO: 18 - DNA, 11354 bp, Artificial Sequence
  pLV-hEF1a-HA-CALCRL-IRES-EYFP-2A-Hygro
ctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagcctggccaccatgaagaccatcctggccctgagcacctacatcttctgcctggtgttcgcctacccctacgacgtgcccgtgtacgccgcagaattagaagagagtcctgaggactcaattcagttgggagttactagaaataaaatcatgacagctcaatatgaatgttaccaaaagattatgcaagaccccattcaacaagcagaaggcgtttactgcaacagaacctgggatggatggctctgctggaacgatgttgcagcaggaactgaatcaatgcagctctgccctgattactttcaggactttgatccatcagaaaaagttacaaagatctgtgaccaagatggaaactggtttagacatccagcaagcaacagaacatggacaaattatacccagtgtaatgttaacacccacgagaaagtgaagactgcactaaatttgttttacctgaccataattggacacggattgtctattgcatcactgcttatctcgcttggcatattcttttatttcaagagcctaagttgccaaaggattaccttacacaaaaatctgttcttctcatttgtttgtaactctgttgtaacaatcattcacctcactgcagtggccaacaaccaggccttagtagccacaaatcctgttagttgcaaagtgtcccagttcattcatctttacctgatgggctgtaattacttttggatgctctgtgaaggcatttacctacacacactcattgtggtggccgtgtttgcagagaagcaacatttaatgtggtattattttcttggctggggatttccactgattcctgcttgtatacatgccattgctagaagcttatattacaatgacaattgctggatcagttctgatacccatctcctctacattatccatggcccaatttgtgctgctttactggtgaatctttttttcttgttaaatattgtacgcgttctcatcaccaagttaaaagttacacaccaagcggaatccaatctgtacatgaaagctgtgagagctactcttatcttggtgccattgcttggcattgaatttgtgctgattccatggcgacctgaaggaaagattgcagaggaggtatatgactacatcatgcacatccttatgcacttccagggtcttttggtgtctaccattttctgcttctttaatggagaggttcaagcaattctgagaagaaactggaatcaatacaaaatccaatttggaaacagcttttccaactcagaagctcttcgtagtgcgtcttacacagtgtcaacaatcagtgatggtccaggttatagtcatgactgtcctagtgaacacttaaatggaaaaagcatccatgatattgaaaatgttctcttaaaaccagaaaatttatataattgagactgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagcaattggagggccgcggcagcctgctgacctgcggcgacgtggaggaaaaccccggccccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactcctggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgagggacaggtgatatccagcacagtggcggccgctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattctgcagatatccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccccctgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttaaatggtacctttaagaccaatgacttacaaggcagctgtagatcttagccactttttaaaagaaaaggggggactggaagggctagctcactcccaacgaagacaagatctgctttttgcttgtactgggtctctctggttagaccagatctgagcctgggagctctctggctgcctagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgacgtgtgtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttattccagtgtggtggaattctgcagtctgc

SEQ ID NO: 19 - DNA, 9749 bp, Artificial Sequence
  pLV-hEF1a-myc-RAMP1-IRES-mKate-2A-Bla
ctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagcctggccaccatgaagaccatcctggccctgagcacctacatcttctgcctggtgttcgccgagcagaagctgatcagcgaggaggacctggcctgccaggaggctaactacggtgccctcctccgggagctctgcctcacccagttccaggtagacatggaggccgtcggggagactctgtggtgtgactggggcaggaccatcaggagctacagggagctggccgactgcacctggcacatggcggagaagctgggctgcttctggcccaatgcagaggtggacaggttcttcctggcagtgcatggccgctacttcaggagctgccccatctcaggcagggccgtgcgggacccgcccggcagcatcctctaccccttcatcgtggtccccatcacggtgaccctgctggtgacggcactggtggtctggcagagcaagcgcactgagggcattgtgtaggactgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacagccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattccgctgagggccgcggcagcctgctgacctgcggcgacgtggaggaaaacccaggcccaatggctaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggataagggacaggtgatatccagcacagtggcggccgctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattctgcagatatccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccccctgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttaaatggtacctttaagaccaatgacttacaaggcagctgtagatcttagccactttttaaaagaaaaggggggactggaagggctagctcactcccaacgaagacaagatctgctttttgcttgtactgggtctctctggttagaccagatctgagcctgggagctctctggctgcctagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgacgtgtgtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttattccagtgtggtggaattctgcagtctgc

SEQ ID NO: 20 - protein, 5 aa, Artificial Sequence
  Synthetic Polypeptide
GSGGS

SEQ ID NO: 21 - protein, 5 aa, Artificial Sequence
  Synthetic Polypeptide
GGGGS

SEQ ID NO: 22 - protein, 4 aa, Artificial Sequence
  Synthetic Polypeptide
GGGS

(19) United States
(12) Patent Application Publication
(10) Pub. No.: US 2015/0018665 A1
(43) Pub. Date: Jan. 15, 2015

(54) MOLECULAR AND CELLULAR IMAGING USING ENGINEERED HEMODYNAMIC RESPONSES

(71) Applicant: Massachusetts Institute of Technology, Cambridge, MA (US)

(72) Inventors: Alan Pradip Jasanoff, Cambridge, MA (US); Adrian Lukas Slusarczyk, Cambridge, MA (US); Mitul Desai, Arlington, MA (US); Mariya Barch, Cambridge, MA (US); Gil G. Westmeyer, Muenchen (DE)

(73) Assignee: Massachusetts Institute of Technology, Cambridge, MA (US)

(21) Appl. No.: 14/332,099

(22) Filed: Jul. 15, 2014

Related U.S. Application Data
(60) Provisional application No. 61/846,232, filed on Jul. 15, 2013.

(51) Int. Cl. A61B 5/02; A61B 5/04; C07K 14/575; A61B 5/145; C12N 9/02; A61K 49/14; A61B 5/00; A61B 5/026

(52) U.S. Cl. CPC A61B 5/02028; A61B 5/0075; A61B 5/4848; A61B 5/04001; A61B 5/026; A61B 5/14546; C12N 9/0075; A61K 49/14; C07K 14/57527; C12N 9/0069; C12Y 113/12007; C07K 2319/61; C07K 2319/50; C07K 2319/41; C07K 2319/42; C07K 2319/22; C07K 2319/60
USPC 600/410; 600/407; 600/479; 530/399; 435/189

(57) ABSTRACT

According to some aspects, the invention relates to methods and compositions for evaluation of hemodynamic responses (e.g., using molecular imaging) with high sensitivity.

### RELATED APPLICATIONS

This application claims priority under 35 U.S.C. §119(e) to U.S. Provisional Application Ser. No. 61/846,232, entitled “MOLECULAR AND CELLULAR IMAGING USING ENGINEERED HEMODYNAMIC RESPONSES” filed on Jul. 15, 2013, which is herein incorporated by reference in its entirety.

### FEDERALLY SPONSORED RESEARCH

This invention was made with government support under Grant Nos. DA028299 and NS076462 awarded by the National Institutes of Health. The government has certain rights in the invention

### BACKGROUND OF INVENTION

A variety of approaches exist for noninvasive functional or molecular physiological imaging. Such methods include, for example, optical microscopy methods as well as positron emission tomography, computed tomography and magnetic resonance imaging. A challenge in molecular imaging is achieving sufficient sensitivity for the molecular targets of interest, which may be present only at very low concentrations. For example, to detect certain molecular targets in the body, over 10 μM of a conventional magnetic resonance imaging (MRI) contrast agent is typically used. Such concentrations may overwhelm endogenous analytes, resulting in perturbations to normal biological function. In certain cases (e.g. certain neurotransmitters in the brain) analytes are too dilute to be detectable by probe concentrations in the micromolar range. Because effective MRI contrast agents are usually polar and large (e.g., >500 Da), in certain instances high concentrations are also particularly difficult to deliver past the blood-brain barrier (BBB), complicating experiments and making clinical applications less plausible.

In addition, certain conditions are a challenge to diagnose using conventional imaging techniques because of the subtlety of associated physiological and microstructural effects. For example, use of conventional computed tomography and MRI scans are often not sufficiently sensitive to structural perturbations present in mild traumatic brain injury (mTBI). Detection of BBB disruption in mTBI is of particular interest. An approach for studying blood brain barrier integrity in patients employs MRI contrast agents such as Gd-DTPA, which are injected intravenously and then accumulate in brain at sites of BBB leakage. Such agents typically need to reach concentrations close to 100 μM to be detected however, and may not escape the vasculature in sufficient quantities to enable detection of subtle BBB disruptions in mTBI.

Accordingly, improved methods for noninvasive functional and molecular physiological imaging are needed.

### SUMMARY OF INVENTION

According to some aspects, the invention relates to methods and compositions for molecular imaging with high sensitivity. In some embodiments, methods and compositions provided herein enable highly sensitive assays for drug testing, basic research, clinical diagnostics and other purposes. In some embodiments, engineered hemodynamic contrast agents, imaging agents and related methods are provided that enable high resolution imaging of molecular and cellular phenomena in organs, tissues and other structures. In some embodiments, agents are provided that produce detectable contrast (e.g., contrast that is detectable using MRI) at submicromolar concentrations, which are 1000-fold or more lower than concentrations associated with conventional contrast agents.

Hemodynamic contrast agents and imaging agents provided herein can be used to enhance the sensitivity of a broad range of conventional imaging techniques including, for example, functional MRI, optical imaging techniques, and others. Because the agents provided herein are useful across a broad range of imaging techniques, direct comparisons can be made among different imaging techniques to enhance confidence in results obtained. This aspect is particularly beneficial in the clinical diagnostic context. Accordingly, in some embodiments, methods and compositions provided herein extend the usefulness of imaging techniques to research and clinical challenges requiring high resolution and sensitive detection of objects or events in vivo.

In some embodiments, imaging methods are provided for detecting physiological abnormalities in organs, tissues and other structures. In some embodiments, methods and compositions are provided that are useful for image-based assessment and mapping of traumatic injury (e.g., traumatic brain injury) phenotypes in clinically relevant populations. In some embodiments, methods and compositions provided here provide engineered hemodynamic contrast that is suitable for detection by techniques that are easy to implement at points of care such as, for example, diffuse optical imaging and photoacoustic tomography methods and others.

Methods provided herein may be used for diagnosing clinical conditions, evaluating drug candidates in humans and in animals, and performing fundamental research in animal models of healthy and diseased physiological function for the ultimate purpose of developing diagnostics and therapeutics. In some embodiments, methods and compositions provided herein are useful for fundamental neuroscience research in pharmaceutical discovery. In some embodiments, methods and compositions provided herein are useful for monitoring of drug action in pharmaceutical development. In some embodiments, methods and compositions provided herein are useful for clinical diagnostics for a broad range of disease conditions and therapeutic regimens.

Some aspects of the invention provide methods for evaluating a tissue in a subject. In some embodiments, the methods involve providing to the tissue an exogenous vasoactive agent in a submicromolar concentration that is effective for inducing a hemodynamic response in the tissue; obtaining an image representation of the tissue; and evaluating the tissue based on a hemodynamic response detected in the image representation. In some embodiments, the methods involve producing a hemodynamic response in a tissue of a subject by providing an effective amount of an exogenous vasoactive agent to the tissue; obtaining an image representation of the tissue; and evaluating the tissue based on a hemodynamic response detected in the image representation. In some embodiments, the methods involve obtaining an image representation of a tissue in a subject; and evaluating the tissue based on a hemodynamic response detected in the image representation, in which the hemodynamic response is induced by an exogenous vasoactive agent.

In some embodiments, the vasoactive agent is provided to the tissue by administering the vasoactive agent to the subject. In some embodiments, administering is performed intravenously. In some embodiments, the vasoactive agent is provided to the tissue by administering the vasoactive agent directly to the tissue. In certain embodiments, the subject is engineered to express the vasoactive agent in the tissue, and the vasoactive agent is provided to the tissue by inducing expression of the vasoactive agent. In some embodiments, the vasoactive agent is provided to the tissue by delivering to the subject a virus comprising a transgene engineered to express the vasoactive agent. In certain embodiments, the virus is engineered to selectively target cells in the tissue. In some embodiments, the transgene is engineered to selectively express the vasoactive agent in the tissue.

In certain embodiments, the image representation is obtained by performing imaging. In some embodiments, the imaging is optical imaging. In certain embodiments, the optical imaging is selected from: photography, reflectance videography, optical endoscopy, brightfield microscopy, confocal microscopy, fluorescence microscopy, optogenetic stimulation and detection, optical coherence microscopy, optical coherence tomography, diffuse optical tomography, near infrared spectroscopy, and Doppler shift-sensitive spectroscopy or imaging. In some embodiments, the imaging is magnetic resonance imaging (MRI). In certain embodiments, the MRI is functional MRI. In some embodiments, the functional MRI is blood-oxygen-level-dependent (BOLD) contrast MRI. In certain embodiments, the imaging is positron emission tomography. In some embodiments, the imaging is infrared thermography, photoacoustic imaging, ultrasonography, echocardiography, or computed tomography.

In certain embodiments, the methods further involve mapping locations of hemodynamic responses within the tissue in the subject based on spatial positioning of hemodynamic responses in the image representation. In some embodiments, the methods further involve obtaining a plurality of image representations of the tissue over time and evaluating spatiotemporal changes hemodynamic responses within the tissue based on differences in the magnitude and/or spatial positioning of hemodynamic responses among the image representations.

In some embodiments, the methods involve obtaining a first image representation of a tissue in a subject; obtaining a second image representation of the tissue; and evaluating the tissue based on hemodynamic responses detected in the first or second image representations, in which the hemodynamics responses are induced by an exogenous vasoactive agent in the tissue, and the first and second image representations are obtained using different imaging techniques.

In certain embodiments, the methods involve detection of hemodynamic responses without explicit image formation. For example, in some embodiments, data may be acquired using optical spectroscopy, magnetic resonance spectroscopy, or other forms of localized spectroscopy and detection of signals related to hemodynamic responses. Measurements may be performed by obtaining a first reading from tissue; obtaining a second reading of the tissue; and evaluating the tissue based on hemodynamic responses detected in the first or second readings.

In some embodiments, measurements may be obtained intermittently or continuously during administration of a vasoactive agent or monitoring of tissue properties.

In some embodiments, the detection of an analyte of interest by a sensor capable of evoking hyperemia by activating a G-protein coupled receptor in vivo can be read out using an in vitro assay of receptor activation. The in vitro assay uses reporter cells (such as yeast, animal, or human cells) that have been engineered to functionally express the relevant receptor, such as a G-protein coupled receptor, and to respond to its activation with a readily detectable signal. In some embodiments, the detectable signal is an optical signal, such as fluorescence, luminescence, or light absorbance. In some embodiments, the reporter cell readout is performed in a microplate and read out using a microplate reader in a laboratory setting. In some embodiments, the reporter cell readout is performed in a lateral flow device.

In certain embodiments, the hemodynamic response comprises a change in blood volume in the tissue. In some embodiments, the hemodynamic response comprises an increase in blood flow in the tissue. In certain embodiments, the hemodynamic response comprises a decrease in blood flow in the tissue. In some embodiments, the hemodynamic response comprises changes in oxy/deoxyhemoglobin balance in the tissue. In certain embodiments, the tissue is neuronal tissue and the hemodynamic response is associated with changes in neural activity.

In some embodiments, the methods involve determining that the hemodynamic response is associated with excitatory neural activity in the tissue. In certain embodiments, the methods involve determining that the hemodynamic response is associated with inhibitory activity in the tissue.

In some embodiments, the vasoactive agent is a small molecule. In certain embodiments, the vasoactive agent comprises lipid moiety. In some embodiments, the vasoactive agent is a protein. In certain embodiments, the vasoactive agent has a molecular mass in a range of 0.5 kDa and 150 kDa.

In some embodiments, the vasoactive agent comprises a detectable moiety. In certain embodiments, evaluating the tissue comprises evaluating a signal emanating from the detectable moiety and relating the signal to a hemodynamic response. In some embodiments, the signal is indicative of a concentration of a physiological analyte; a catalytic activity; or a biological activity. In certain embodiments, the biological activity is gene expression; protein secretion; protein modification; receptor activation; pathway activation; pathway inhibition; exocytosis; endocytosis; or vesicle cycling.

In some embodiments, the vasoactive agent modulates hemodynamic response through activation of one or more endogenous biochemical pathways in cells of the tissue. In certain embodiments, the effective amount of the vasoactive agent is a submicromolar concentration of the vasoactive agent in the tissue. In some embodiments, the vasoactive agent produces an artificial hemodynamic response in the tissue. In certain embodiments, the vasoactive agent is a vasoactive neuropeptide that is secreted by endogenous cells, for example neurons. In some embodiments, the vasoactive agent effects dilation of the microvasculature. In certain embodiments, the vasoactive agent binds to a heterodimeric, G-protein coupled receptor on the surface of cells. In some embodiments, the vasoactive agent is a vasoactive neuropeptide that translates molecular signals into engineered hyperemia. In certain embodiments, the vasoactive agent is nitric acid synthase (NOS) or an engineered derivative thereof. In some embodiments, the vasoactive agent is calcitonin gene-related peptide (CGRP) or an engineered derivative thereof. In certain embodiments, the vasoactive agent is an engineered protein that is conditionally active. In some embodiments, the protein is activated by interacting with an endogenous analyte in the tissue.

In certain embodiments, the vasoactive agent is a fusion protein comprising a neurotransmitter analog, CGRP, and a binding domain such that the interaction between the binding domain and the analog disrupts CGRP function. In some embodiments, the vasoactive agent is an engineered chimera of a catalytic domain of inducible NOS (NOS) and a regulatory domain of NOS. In certain embodiments, activity of the engineered chimera is dependent on calcium bound calmodulin, and independent on a blood brain barrier (BBB)-permeable inhibitor with specificity for an endogenous NOS catalytic domain. In some embodiments, the BBB-permeable inhibitor is 7-nitroindazole (7-NI).

In certain aspects of the invention, methods are provided for evaluating signaling of a biochemical pathway in a tissue. In some embodiments, the methods involve providing to a tissue a vasoactive agent, the activity of which vasoactive agent is modulated by signaling of a biochemical pathway in the tissue, in which modulation of the vasoactive agent results in a hemodynamic response in the tissue; and evaluating signaling of the biochemical pathway in the tissue by assessing in an image representation of the tissue presence or absence of a hemodynamic response resulting from modulation of the vasoactive agent in the tissue. In some embodiments, the signaling is calcium signaling. In some embodiments, the signaling is indicative of catalytic activity of matrix metalloproteases involved in tumor metastasis. In some embodiments, the signaling is indicative of catalytic activity of Fibroblast Activating Protein involved in tumor invasion and metastasis. In some embodiments, the signaling is indicative of catalytic activity of caspases involved in apoptosis. In some embodiments, the vasoactive agent is an engineered protein. In some embodiments, the engineered protein is activated by a calcium-binding protein that is present in the tissue, in which activation of the engineered protein results in a hemodynamic response in the tissue. In some embodiments, the methods further involve obtaining an image representation of the tissue.

In some aspects of the invention, methods are provided for evaluating calcium signaling in a tissue. In some embodiments, the methods involve providing to a tissue an engineered protein that is activated to induce a hemodynamic response in the tissue through interactions with a calcium-binding protein; inhibiting activity of an endogenous protein that induces the hemodynamic response in the tissue; and evaluating calcium signaling in the tissue by assessing presence or absence of the hemodynamic response resulting from activation of the engineered protein in the tissue. In some embodiments, the engineered proteins comprises a catalytic domain of inducible NOS (iNOS) fused to a regulatory domain of NOS. In some embodiments, the calcium binding protein is calmodulin, and wherein calcium-bound calmodulin activates the engineered protein. In some embodiments, inhibiting activity of an endogenous protein comprises delivering to the tissue an inhibitor of endogenous NOS activity in the tissue. In some embodiments, presence or absence of the hemodynamic response is assessed by obtaining an image representation of the tissue and assessing the image representation for the presence or absence of the hemodynamic response in the tissue. In some embodiments, the methods further involve stimulating signaling of the biochemical pathway in the tissue or calcium signaling in the tissue by subjecting the subject to a physical or behavioral task. In some embodiments, the methods further involve delivering to the tissue an agent that stimulates signaling of the biochemical pathway in the tissue or calcium signaling in the tissue. In some embodiments, the vasoactive agent or engineered protein is provided to dopaminergic neurons in the tissue.

In some aspects of the invention, methods are provided for assessing blood-brain barrier (BBB) integrity. In some embodiments, the methods involve providing to the vasculature of a subject an exogenous vasoactive agent that is impermeable to the BBB; obtaining an image representation of brain tissue; and assessing BBB integrity by evaluating the image representation for the presence or absence of a hemodynamic response induced by the exogenous vasoactive agent. In some embodiments, the exogenous vasoactive agent is calcitonin gene-related peptide (CGRP). In some embodiments, the hemodynamic response is a vasodilatory effect in the brain vasculature, and detection of the hemodynamic response in the image representation indicates disruption of the BBB. In some embodiments, the methods further involve detecting the hemodynamic response and determining that disruption of the BBB is a result of a traumatic injury, carcinogenesis or inflammation.

In some aspects of the invention, methods are provided for evaluating ligand binding in a tissue. In some embodiments, the methods involve providing to a tissue a vasoactive agent, the activity of which vasoactive agent is modulated by binding to a ligand, in which modulation of the vasoactive agent results in a hemodynamic response in the tissue; obtaining an image representation of the tissue; and evaluating ligand binding by assessing in the image representation presence or absence of a hemodynamic response resulting from modulation of the vasoactive agent in the tissue. In some embodiments, a compound is provided that comprises a calcitonin gene-related peptide (CGRP) fused to an analyte binding molecule that inhibits activity of CGRP in the absence of analyte. In some embodiments, a compound is provided that comprises a calcitonin gene-related peptide (CGRP) fused to an analyte binding domain and inhibitory domain, such that the compound is vasoactive in the presence of an analyte which disrupts intramolecular interactions between the binding and inhibitory domains.

In some embodiments, a compound is provided that comprises a calcitonin gene-related peptide (CGRP) fused to a second peptide, wherein the compound is vasoactive following enzymatic modification of the peptide. In some embodiments, the enzymatic modification is cleavage of the second peptide. In some embodiments, the enzymatic modification is acetylation, acylation, adenylylation, ADP-ribosylation, carbamylation, deamidation, deamination, glycation, glycosylation, hydroxylation, imine formation, methylation, myristoylation, o-glycosylation, palmitoylation, phosphorylation, prenylation, sulfation, sumoylation, transglutamination, or ubiquitination. In some aspects, compositions are provided that comprise a compound disclosed herein and a physiologically acceptable carrier. In some aspects, kits are provided that comprise one or more containers housing a compound or composition disclosed herein. According to some aspects, methods are provided that involve providing a compound disclosed herein to a tissue; obtaining an image representation of the tissue; and determining presence or absence of a hemodynamic response in the tissue based on the image representation.

According to some aspects, methods are provided that involve introducing an imaging agent precursor to a subject; via a reaction involving the precursor, effecting production, in the subject, of an imaging agent; and imaging tissue via the agent. In some embodiments, the reaction involves at least the precursor and a physiological species. In some embodiments, the imaging agent is produced at a concentration at least five times the concentration of the agent. In some embodiments, the reaction involving the precursor occurs in the imaged tissue. In some embodiments, the imaging agent precursor is introduced at a concentration of 0.0001 to 100 mg/kg, 0.01 to 100 mg/kg, 0.001 to 10 mg/kg, or 0.0001 to 10 mg/kg of body weight, e.g., about 100, 10, 1, 0.1, 0.01, 0.001, or 0.0001 mg per kg of body weight of the subject.

According to some aspects, methods are provided that involve introducing a bioactive agent to a subject; via a reaction involving the bioactive agent, effecting an detectable alteration of the quantity of an imaging agent in a tissue in the subject; and imaging the tissue via the imaging agent. In some embodiments, the bioactive agent is a vasoactive agent. In some embodiments, the imaging agent is deoxyhemoglobin or oxyhemoglobin. In some embodiments, the reaction effects a dilation or constriction of vasculature in the tissue.

According to some aspects, methods are provided that involve introducing a bioactive agent to a subject; via a reaction involving the bioactive agent, effecting an detectable alteration of endogenous contrast in the subject; and imaging tissue based on the endogenous contrast. In some embodiments, the bioactive agent is a vasoactive agent. In certain embodiments, the endogenous contrast is a diffusion pattern of water and/or other molecules through an organ or tissue structure. In some embodiments, the organ is a brain. In some embodiments, the tissue structure is vasculature. In some embodiments, the imaging is performed using diffusion-weighted MRI.

According to some aspects, engineered vasoactive agents are provided that have the formula X¹-L¹-X²-L²-X³, in which X¹ is a blocking domain and/or ligand binding domain, L¹ and L² are independently linkers or absent, X² is a protease recognition site and/or a ligand or analog thereof, and X³ is a vasoactive molecule (e.g., as depicted in FIG. 2F). According to some aspects, engineered vasoactive agents are provided that have the formula X¹-L-X²-X³ or X¹-X²-L-X³, in which X¹ is a blocking domain and/or ligand binding domain, L is a linker, X² is a protease recognition site and/or a ligand or analog thereof, and X³ is a vasoactive molecule (e.g., as depicted in FIG. 2F). In some embodiments, X¹ is a blocking domain, L is a linker, X² is a protease recognition site, and X³ is a vasoactive molecule. In some embodiments, X¹ is a ligand binding domain, L is a linker, X² is a ligand or analog thereof, and X³ is a vasoactive molecule. In some embodiments, engineered vasoactive agents are provided that have the formula X¹-X² or X¹-L-X², in which X¹ is one or several repeats of an exopeptidase cleavage sequence, L is a linker, and X² is a vasoactive molecule. In some embodiments, X¹ here serves a dual purpose as a blocking domain and protease cleavage site. In some embodiments, engineered vasoactive agents are provided that have the formula X¹-L¹-X²-L²-X³, where X¹ is an N-terminal subsequence of a vasoactive peptide, L¹ and L² are linkers, X² is an allosteric ligand binding domain, and X³ is a C-terminal subsequence of a vasoactive peptide. Many additional embodiments that combine sensing domains and vasoactive domains are contemplated; in some aspects, such molecules are constructed using synthetic chemical building blocks as opposed to peptides or other biopolymers.

### BRIEF DESCRIPTION OF DRAWINGS

FIG. 1A depicts a non-limiting example of a process flow chart for functional molecular imaging;

FIG. 1B depicts a non-limiting example of a process flow chart for functional molecular imaging in the context of organism scale synthetic biology;

FIGS. 2A-2F depict a non-limiting example of a cell-based bioassay that reports activation of the CGRP receptor;

FIG. 2A shows activation of the heterodimeric CGRP receptor elevates intracellular cAMP; in vascular smooth muscle cells, cAMP inhibits myosin light chain kinase and leads to muscle relaxation and vasodilation; in engineered HEK293FT reporter cells, cAMP allosterically activates an engineered luciferase;

FIG. 2B shows the lentiviral DNA constructs used to generate HEK293FT CGRP reporter cells;

FIG. 2C shows, after antibiotic selection, the reporter cells express the fluorescent markers associated with each lentiviral construct; FIG. 2D shows dose-response curves with synthetic CGRP (Sigma) for HEK293FT CGRP reporter cells and for HEK293FT cells transduced with only the luciferase-bearing lentivirus show receptor-dependent cAMP elevation in response to subnanomolar CGRP concentrations;

FIG. 2E shows receptor activation by CGRP is specifically inhibited by [8-37]CGRP, which is atruncated form of wildtype human alpha CGRP comprising amino acids 8 through 37, acting as a competitive inhibitor of CGRP agonist activity;

FIG. 2F depicts non-limiting examples of CGRP-based sensors;

FIGS. 3A-D depict a non-limiting example of an engineered hemodynamic response induced by CGRP injection;

FIG. 3A shows an optical image of exposed cortex highlighting vascular regions of interest (ROIs);

FIG. 3B shows percent signal changes over time (x-axis) for ROIs outlined in FIG. 3A; 10 nM CGRP was infused during the shaded time period;

FIG. 3C shows an MRI map showing areas of significant signal change (colored voxels) due to 500 nM CGRP infusion through a cannula in the left hemisphere; control infusion of cerebrospinal fluid on the right side produced no significant changes; FIG. 3D show quantified group data (n=4);

FIGS. 4A-D are non-limiting examples of the effects of terminal modifications on CGRP agonist activity;

FIG. 4A shows primary and secondary structure of CGRP (SEQ ID NO: 1). FIG. 4B shows that after incubation with 10 mM DTT, the potency of CGRP is reduced due to opening of the N-terminal disulfide ring;

FIG. 4C shows the replacement of the C-terminal amide with carboxylic acid reduced potency by 3 orders of magnitude; extension with a terminal glycine partially restores potency;

FIG. 4D shows N-terminal extension of CGRP by Gly or IleAlaGly has a much smaller effect on potency than C-terminal alterations;

FIGS. 5A-D is a non-limiting example of molecular sensors based on CGRP;

FIG. 5A shows schematic architecture of CGRP-based protease sensors with cleavage sites for (top to bottom) MMP-9, caspase-3, Factor Xa, TEV protease, and enterokinase; MSAWSHPQFEKGA (SEQ ID NO: 2); GGSG (SEQ ID NO: 13); GPLGIAG, DEVD, IEGR, ENLYFQG, DDDDK (SEQ ID NOs: 8-12, respectively).

FIG. 5B shows a caspase sensor incubated in the presence or absence of caspase-3;

FIG. 5C depicts MALDI spectrum showing cleavage of caspase sensor;

FIG. 5D shows sensors for caspase-3, enterokinase and TEV protease show bioactivity after proteolytic removal of the GFP blocking domain;

FIG. 6 depicts a non-limiting example of engineered nitric oxide synthase (Chi-1); and

FIG. 7 depicts a non-limiting example of Chi-1 enzymatic activity; results were normalized to the maximum nitrate formation on that day and were from 3 independent transfection experiments; control refers to transfection reagent only; iono refers to calcium ionophore antibiotic a23187 at 5 μm; nNOS refers to rat nNOS; and Chi 1 refers to engineered nNOS.

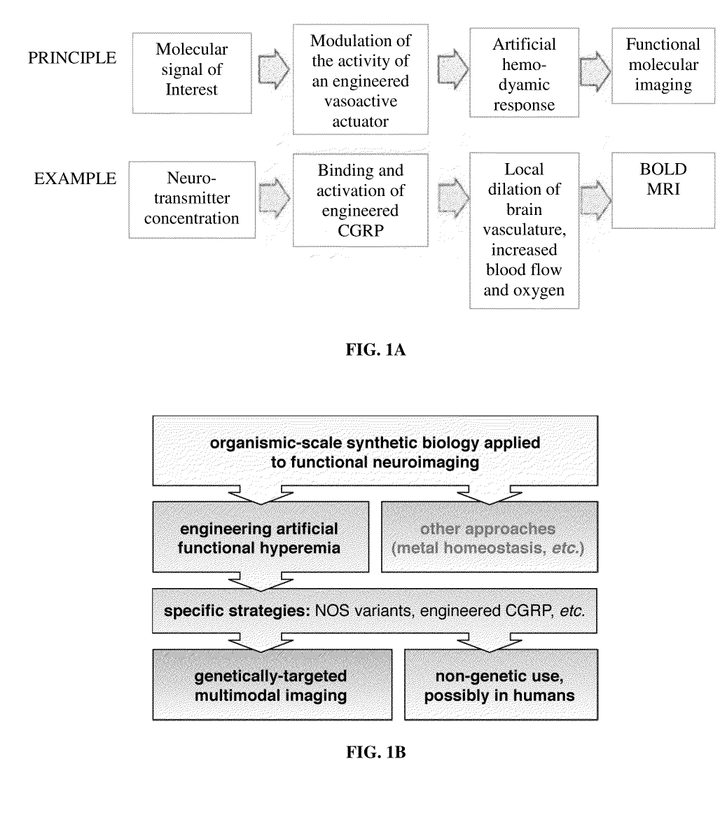

### DETAILED DESCRIPTION OF INVENTION

Provided herein are methods for molecular imaging with high sensitivity. Methods provided herein are useful in a broad range of areas, including, but not limited to, biomedical research, target discovery, as well as drug screening, development, and characterization. In some embodiments, methods provided herein are useful for gaining a functional physiological understanding of mechanisms underlying major disease areas. Accordingly, in some embodiments, use of methods provided herein may lead to the discovery of addressable functional mechanisms in health and disease. In some embodiments, methods provided herein may be used to assess pharmacological or pharmacokinetic properties of drugs (including lead molecules) based on molecular imaging.

Certain methods provided herein are useful for evaluating an organ, tissue or other structure in a subject. In some embodiments, methods provided herein involve providing to an organ, tissue or other structure in a subject an exogenous vasoactive agent for purposes of inducing a hemodynamic response. In some embodiments, the exogenous vasoactive agent is provided in a submicromolar concentration (e.g., 1 nM to less than 1 μM, 1 pM to less than 1 μM, 1 pM to 500 nM, 1 nM to 100 nM, 10 nM to 200 nM, 50 nM to 500 nM, 1 nM to less than 1 μM) that is effective for inducing a hemodynamic response in the tissue. In some embodiments, methods provided herein further involve obtaining an image representation of an organ, tissue or other structure and evaluating the organ, tissue or other structure based on a hemodynamic response detected in the image representation.

As used herein, the term “subject” refers to any animal, such as a mammal, including but not limited to a human, non-human primate, rodent (e.g., mouse, rat), pig, guinea pig, rabbit, cat, dog, goat, cow, horse, etc.

As used herein, the term “hemodynamic response” refers to an alteration of blood vessel dilation and/or blood flow and/or blood pressure and/or oxygenation level of blood in a subject.

As used herein, the term “vasoactive agent” refers to any agent that effects a constriction or dilation of a blood vessel, and/or that modulates blood pressure and/or heart rate in a subject. A vasoactive agent can bring about vasodilation or vasoconstriction. A vasoactive agent may be a natural or synthetic molecule. A vasoactive agent may be a small molecule, such as, for example, nitric oxide (NO), small molecules that promote generation of NO, prostaglandins, eicosanoids, cytochromes, ATP, analogues of ATP, or other small molecules. A vasoactive agent may comprise a protein or peptide, such as nitric oxide synthase, cyclooxygenase (COX), Calcitonin gene-related peptide (CGRP) and others.

In some embodiments, a vasoactive agent is a molecule having the formula X¹-L-X²-X³ or X¹-X²-L-X³, in which X¹ is a blocking domain and/or ligand binding domain, L is a linker, X² is a protease recognition site and/or a ligand or analog thereof, and X³ is a vasoactive molecule. In some embodiments, a vasoactive agent is a molecule having the formula X¹-L-X²-X³, in which X¹ is a blocking domain, L is a linker, X² is a protease recognition site, and X³ is a vasoactive molecule. In some embodiments, a vasoactive agent is a molecule having the formula X¹-L-X²-X³, in which X¹ is a ligand binding domain, L is a linker, X² is a ligand or analog thereof, and X³ is a vasoactive molecule. In some embodiments, engineered vasoactive agents are provided that have the formula X¹-X² or X¹-L-X², in which X¹ is one or several repeats of an exopeptidase cleavage sequence, L is a linker, and X² is a vasoactive molecule. X¹ here serves a dual purpose as a blocking domain. In some embodiments, engineered vasoactive agents are provided that have the formula X¹-L¹-X²-L²-X³, where X¹ is an N-terminal subsequence of a vasoactive peptide, L¹ and L² are linkers, X² is an allosteric ligand binding domain, and X³ is a C-terminal subsequence of a vasoactive peptide.

With regard to L, any suitable linker may be used, including, for example, polypeptide and non-polypeptide linkers. In some embodiments, polypeptide linkers may comprise small flexible amino acids such as Gly, Ser, Ala and Thr. In one embodiment the linker comprises or consists of glycine residues. In one embodiment the linker comprises or consists of serine residues. In one embodiment the linker comprises or consists of alanine residues. In one embodiment the linker comprises or consists of threonine residues. In one embodiment the linker comprises or consists of glycine and serine residues. In one embodiment the linker comprises or consists of glycine, serine and alanine residues. In one embodiment the linker comprises or consists of glycine, serine, alanine and threonine residues. It should be appreciated understood that all permutations of glyine and/or serine and/or alanine and/or threonine residues may be used in some embodiments. In one embodiment, a linker comprises or consists of 30-80% glycine residues and 20-70% serine residues. In one embodiment, the linker comprises or consists of 35-50% glycine residues; 30-40% serine residues; 5-15% threonine residues and 10-20% alanine residues. In one embodiment, the amino acid residues are randomly distributed within the linker. Specific examples of suitable linkers include glycine-serine polymers comprising for example repeats of sequences such as GS, GGS, GGSG (SEQ ID NO: 13), GSGGS (SEQ ID NO: 20), GGGGS (SEQ ID NO: 21) and GGGS (SEQ ID NO:22). In some embodiments, polypeptide linkers may be 1 to 5 amino acids, 1 to 10 amino acids, 5 to 20 amino acids, 10 to 30 amino acids, 10 to 40 amino acids, 20 to 50 amino acids, 30 to 100 amino acids or more.

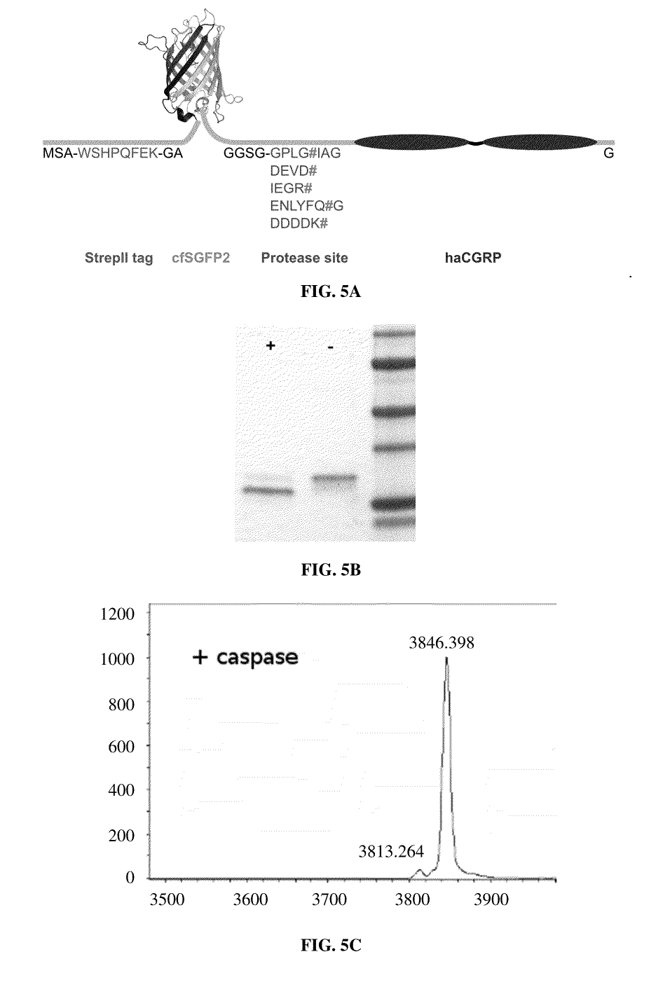

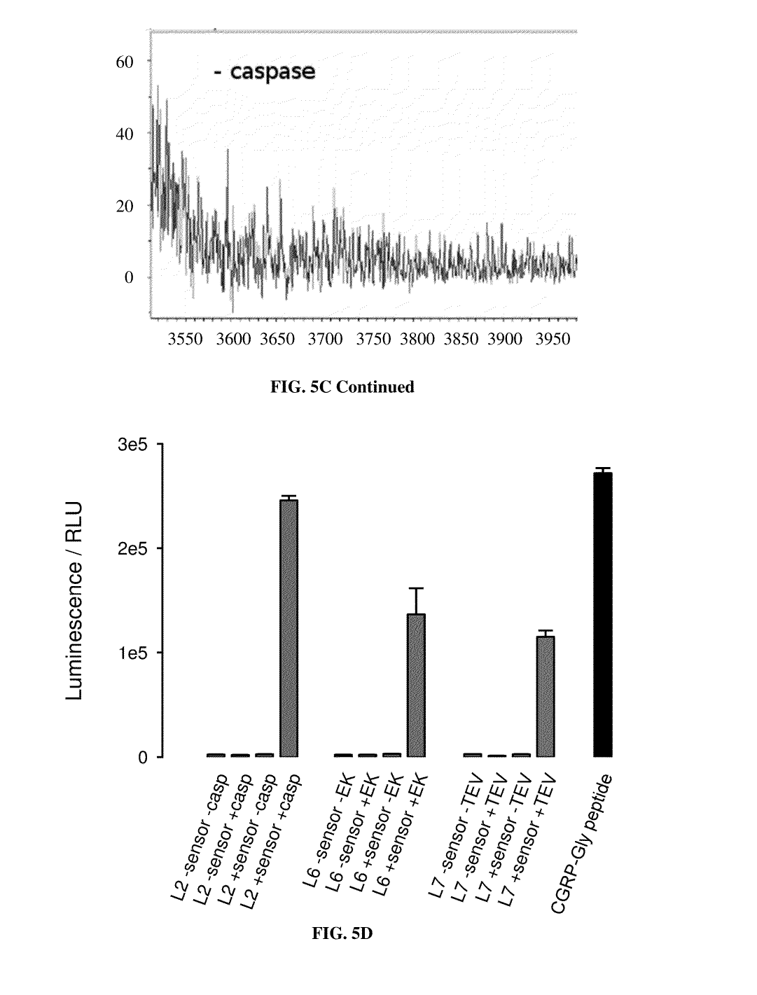

In embodiments in which X² comprises a protease recognition site, any suitable protease recognition site may be used. In some embodiments, the protease recognition site is a recognition site of a serine protease or a serine-threonine protease. In some embodiments, the protease recognition site is a recognition site of a matrix metalloproteinase, such as MMP-9. In some embodiments, the protease recognition site is a recognition site of a caspase, such as caspase-3. In some embodiments, the protease recognition site is a recognition site of an endopeptidase, such as Tobacco Etch Virus (TEV) protease or Factor Xa. Further non-limiting examples of suitable protease recognition sites are provided in FIG. 5A.

In embodiments in which X² comprises a ligand, the ligand may bind selectively to a ligand binding protein of X¹. In some embodiments, a vasoactive peptide of X³ is unable to effect a hemodynamic response when a ligand of X² is bound to a ligand binding protein of X¹ in the absence of soluble ligand. However, in some embodiments, in the presence of soluble ligand, the soluble ligand competes for binding to the ligand binding protein with the tethered ligand of X², thereby relieving the inhibitory effect on the vasoactive peptide, and permitting the vasoactive peptide to induce a hemodynamic response. In some embodiments, X² comprises a neurotransmitter or analog thereof that binds to a neurotransmitter binding protein of X¹, allowing the vasoactive agent to function as a sensor for the presence of soluble neurotransmitter. In some embodiments, a neuroactive peptide (e.g., CGRP, Max, ADM) is conjugated to a tethered neurotransmitter analog and to a neurotransmitter binding protein in such a way that intramolecular binding between the tethered analog and binding domain reversibly disrupts peptide's ability to induce vasodilation.

In embodiments in which X¹ comprises a blocking protein, the blocking protein is typically configured to block, reduce or inhibit the ability of the vasoactive molecule of X³ to effect a hemodynamic response. In some embodiments, cleavage of a protease recognition site of X² separates the blocking protein of X¹ from the vasoactive molecule of X³, thereby relieving the inhibition. In some embodiments, the blocking protein is a globular protein. In some embodiments, the blocking protein is an enzyme, messenger protein, or structural protein. In some embodiments, the blocking protein is a hemoglobin or other member of the globin protein family. In some embodiments, the blocking protein is an immunoglobulins (e.g., IgA, IgD, IgE, IgG and IgM) or a fragment thereof. In some embodiments, the blocking protein is an alpha, beta or gamma globulin. In some embodiments, the blocking protein is a fluorescent protein, such as, for example, a green fluorescent protein or variant thereof.

With regard to X³, any suitable vasoactive molecule may be used, including, for example, a vasoactive peptide. In some embodiments a vasoactive peptide is selected from the group consisting of: calcitonin gene-related peptide (CGRP), human atrial natriuretic peptide (hANP), endothelin (ET), adrenomedullin (ADM), Maxadilan (Max), pituitary adenylate cyclase-activating polypeptide (PACAP), vasoactive intestinal peptide (VIP), peptide histidine isoleucine (PHI), peptide histidine methionine (PHM), peptide histidine valine (PHV), peptide N-terminal histidine C-terminal methionelmide, angiotensin I, angiotensin II, bombesin, cholecystokinin-pancreozymin, neurotensin, oxytocin, prolactin, sauvagine, somatostatin, substance P, opioid peptides, relaxin, amylin, thyrotropin-releasing hormone, parathyroid hormone, urotensin, histamine, endothelium-derived hyperpolarizing factor (EDHF), vasopressin and related natural and synthetic analogs of any of them. In some embodiments a vasoactive peptide is a tachykinin, such as, for example, neurokinin A, bradykinin, neuokinin B, and others. In some embodiments a vasoactive peptide is calcitonin gene-related peptide (CGRP) or an analog thereof. In some embodiments a vasoactive peptide is adrenomedullin (ADM), or an analog thereof. In some embodiments a vasoactive peptide is Maxadilan (Max) or an analog thereof.

In embodiments in which X¹ represents one or several repeats of an exopeptidase cleavage sequence, X¹ simultaneously serves as a blocking domain attenuating the potency of the vasoactive moiety X² while X¹ is present such that removal of X¹ by one or several repeated exopeptidase cleavage events restores the potency of X². For example, X² can be the amino acid sequence APAP which is removed by two cleavage events mediated by the alanyl-prolyl exopeptidase activity of Fibroblast Activation Protein (FAP).

In embodiments in which X² represents an allosteric ligand binding domain inserted into the middle of a vasoactive agent, any naturally occurring or engineered ligand binding domain that is capable of allosterically modulating the agonist activity of the vasoactive moiety is suitable. For example, bacterial Periplasmic Binding Proteins may be used to engineer fluorescent biosensors by allosterically modulating fluorescent reporter fusions. In some embodiments, members of the same family of protein domains may be employed to alter the spatial conformation of a vasoactive moiety, such as Maxadilan in such way that its receptor agonist activity (and thereby its vasoactive property) is attenuated or enhanced in the presence of a ligand of interest. Other suitable sources of ligand binding domains include naturally occurring receptors for ligands of interest, such as neurotransmitter receptors, or engineered variants thereof. Those skilled in the art will appreciate that similar sensor architectures may be assembled using small molecules or other nonbiosynthetic or biosynthetic building blocks.

As used herein, the term “image representation” refers to any depiction of the spatial organization or arrangement of one or more objects or events. In some embodiments, an image representation is a two-dimensional depiction. In some embodiments, an image representation is a three-dimensional depiction. Examples of image representations include, but are not limited to, images, photographs, videos, x-rays, microfiche, microfilm, or any other recordings or depictions of the physical appearance or arrangement of any object or event by any technique. In some embodiments, an image representation can be produced from any data containing positional or spatial information. For example, certain recording techniques, such as electroencephalography (EEG), magnetoencephalography (MEG), electrocardiography (EKG), which produce data containing positional information, can be used to produce an image representations of the data (e.g., image representations of electrical activity). Image representations encompass any digital image or video retrievable from computer storage.

In some embodiments, molecular imaging methods are provided herein that involve measuring and/or visualizing molecular concentrations or activities in vivo. In some embodiments, cellular imaging methods are provided herein that involve measuring and/or visualizing aspects of cellular function. In some embodiments, methods provided herein involve relating signals of interest (e.g., molecular concentrations or activities, or aspects of cellular function) with a hemodynamic response.

In some embodiments, methods provided herein are useful for clinical diagnostic imaging. In such embodiments, methods provided herein may involve molecular imaging of a hemodynamic response to assess molecular concentrations or activities. In some embodiments, molecular imaging of hemodynamic responses is used to assess concentrations or activities of neurotransmitters. In some embodiments, molecular imaging of hemodynamic responses is used to assess concentrations or activities of factors associated with disease. In some embodiments, image-based detection of hemodynamic responses using methods provided herein facilitates the study of drug effects in vivo. In some embodiments, image-based detection of hemodynamic responses is useful for detecting disruptions to blood brain barrier due to traumatic brain injury, inflammation, carcinogenesis or other conditions.

In some embodiments, methods are provided for assessing functional hyperemia in organs, tissues or other structures in a subject. As used herein, the term “functional hyperemia” refers to a hemodynamic response to increased neural activity involving increases in blood flow. In some embodiments, increases in blood flow are triggered by elevations in neural activity levels during stimuli or behavioral tasks. In some embodiments, blood flow increases associated with functional hyperemia lead to changes in cerebral blood volume and oxy/deoxyhemoglobin balance.

In some embodiments, methods provided herein may be used to visualize tissue structures and/or boundaries. In some embodiments, where a structure of interest is the brain, any suitable modality of imaging of brain tissue may be used. For example, optical imaging of blood vessel dilation or magnetic resonance imaging of the changes in magnetic properties of blood which accompany changes in blood oxygenation level may be used for purposes of translating molecular or cellular phenomena of interest into a hemodynamic response.

In some embodiments, methods are provided herein that involve functional brain imaging for assessing hemodynamic phenomena. In some embodiments, methods provided herein are particularly useful because they capture information about the specificity of underlying neuronal activity, which is often lost using conventional approaches. For example, methods provided herein may be used to distinguish hemodynamic contributions arising from excitatory vs. inhibitory activity. In some embodiments, methods provided herein utilize magnetic resonance imaging (MRI) to detect and/or monitor functional hyperemia via a BOLD effect in which a brightening of tissue MRI signal results from decreased deoxyhemoglobin during blood flow increases. In some embodiments hyperemic responses may be detected using vascular MRI contrast mechanisms such as dynamic arterial spin labeling, cerebral blood volume imaging, and other methodologies sensitive to hemodynamics.

In some embodiments, hyperemic responses are also assessed using optical imaging techniques. Optical techniques include but are not limited to light microscopy, reflectance imaging, confocal microscopy, multiphoton microscopy, superresolution microscopy, diffuse optical tomography, near infrared spectroscopy and imaging, and diffuse fluorescence or luminescence imaging. In some embodiments, detection of hyperemic responses using optical imaging techniques achieve higher temporal resolution than certain MRI based techniques. In some embodiments, hyperemic responses are assessed using imaging techniques based on ultrasound, nuclear medicine, or other imaging techniques. These embodiments include but are not restricted to ultrasound imaging, photoacoustic tomography, positron emission tomography, single photon emission computed tomography, X-ray computed tomography. Those skilled in the art will appreciate in what manner each of these detection methods can be applied to detect hyperemia or and other aspects of blood flow.

In some embodiments, methods are provided herein for functional imaging in vivo of tissue and organs (including, but not limited to, the brain). In some embodiments, a functionally relevant molecular or cellular phenomenon is detected using an engineered moiety and relayed into a hemodynamic response, which is then externally detected and recorded. In some embodiments, the concentration of a physiological analyte is detected. In some embodiments, catalytic activity of an enzyme is detected. In some embodiments, a biological activity, such as gene expression or secretion is detected. In some embodiments, a molecular entity (e.g., a synthetic or engineered biological molecule) effects a hemodynamic response upon detection of a signal of interest (e.g., calcium signaling) through activation of endogenous pathways.

In some embodiments, methods provided herein involve implementation of functional brain imaging using optical imaging or BOLD MRI as a readout.

In some embodiments, biochemical pathways that regulate natural hemodynamics with nanomolar sensitivity to endogenous modulators, are modulated using exogenous agents (e.g., exogenous vasoactive agents) to achieve artificial hemodynamic responses.

In some embodiments, engineered vasoactive neuropeptides are used for translating molecular signals into engineered hyperemia. In some embodiments, engineered nitric acid synthase (NOS) is used for translating molecular signals into engineered hyperemia.

In some embodiments, methods provided herein may be used to visualize spatial and/or temporal patterns of gene expression. In some embodiments, methods provided herein may be used to detect of physiologically relevant molecular species. In some embodiments, methods provided herein may be used to detect catalytic activity.

In some embodiments, hyperemic responses induced by vasoactive agents or probes could also be detected by non-imaging methods. Such methods may be based on visual inspection (e.g. of vasodilation in skin), or by noninvasive readouts. Noninvasive readouts include pulse oximetry, in vivo spectroscopy at near infrared or other wavelengths, and non-imaging analogs of ultrasound, photoacoustic tomography, magnetic resonance, X-ray, nuclear medicine, endoscopy, and other modalities commonly used for medical analysis. In some embodiments, hyperemic responses may be detected by conventional photography and analysis of photographic images. In some embodiments, detection may be performed using portable devices, such as devices commonly used for medical monitoring in clinical settings. Such devices may include pulse oximetry devices, Doppler ultrasound devices, endoscopic devices, or other devices used for invasive photonic or chemical detection. In some embodiments, devices suitable for detecting hyperemia, blood flow, or vascular structure may be employed.

In some embodiments, detection of an analyte of interest by a sensor capable of evoking hyperemia by activating a receptor, such as a G-protein coupled receptor, in vivo can be read out using an in vitro assay of receptor activation. In some embodiments, the ex vivo assay of GPCR activation is a microplate assay using reporter cells which functionally express the relevant receptor. In some embodiments, the reporter cells are human cells. In some embodiments, the reporter cells are non-human animal cells. In some embodiments, the reporter cells are yeast cells. In some embodiments, the reporter cells respond to receptor activation by producing an optical signal, for example but not limited to fluorescence, luminescence, or absorbance. Such an assay may be employed as a laboratory diagnostic test for an analyte of interest in a biological sample. In some embodiments, an analyte of interest can be a clinical diagnostic biomarker. In some embodiments, an analyte of interest may be an enzyme (examples of which are given elsewhere in this patent). In some embodiments, an analyte of interest may be a ligand (examples of which are given elsewhere in this patent). In some embodiments, an analyte of interest may be Prostate Specific Antigen. In some embodiments, an analyte of interest may be a pathogen-associated biomarker. In some embodiments, the biological sample may be urine, blood, saliva, tissue extract, or any patient-derived fluid.

In some embodiments, the in vitro assay of receptor (e.g., G-protein coupled receptor) activation by an analyte-responsive vasoactive sensor can be a lateral flow assay. In the lateral flow assay, reporter cells (such as yeast cells) functionally expressing the relevant receptor and capable of producing an optical readout upon receptor activation are deposited in a matrix capable of fluid transport. A biological sample (such as a patient-derived fluid or a fluid prepared through additional steps from a patient sample) is placed onto the matrix and allowed to flow into contact with the reporter cells. Then, an optical readout of receptor activation is performed. Devices implementing such an in vitro readout of the detection of an analyte of interest by engineered switchable vasoactive sensors may be employed. In some embodiments, the use of engineered switchable receptor agonists in conjunction with a device and method for their in vitro readout could serve as a point-of-care medical diagnostic test.

### Sensitive and Functionally-Specific Measures of Brain Activity.

In some embodiments, methods are provided in which the brain is engineered to report precise aspects of its own function, in real time, and at a comprehensive spatial scale in conjunction with noninvasive imaging-based detection. In contrast to conventional molecular neuroimaging approaches, which have typically focused on individual chemical or biomolecular probes, sensitive and specific neuroimaging readouts are established herein by purposefully perturbing endogenous biological systems, which in some embodiments are related to neurovascular coupling in the brain.

In some embodiments, methods provided herein addresses several challenges in neuroscience and neuroimaging. In some embodiments, by facilitating noninvasive analysis of molecular-level neurophysiology, measurements provided herein facilitate assessment of molecular signaling events across a whole brain. In some embodiments, a wide array of neural phenomena are detected using components that are targeted via genetic or non-genetic approaches. In some embodiments, by engaging and manipulating biological processes that are readily detected using methods such as magnetic resonance imaging (MRI) and diffuse optical tomography (DOT), improvements in sensitivity of molecular measurements are achieved. In some embodiments, by improving sensitivity of in vivo molecular imaging, use of invasive delivery of substantial amounts of exogenous imaging agents or use of radioactive tracers is obviated, reducing potential toxicity and unwanted interactions with cells. Accordingly, in some embodiments, methods provided herein facilitate real-time molecular neuroimaging using multiple modalities in human subjects. In some embodiments, specific methods are provided by which endogenous hemodynamics is utilized to support molecular imaging of multiple neural activity components without the use of actual molecular imaging agents.

In some embodiments, methods provided herein enable monitoring of brain function with molecular specificity across entire brains and provide information about the cellular nature of underlying neuronal activity and mechanistic information about brain function. In some embodiments, molecular imaging agents are provided that are sensitive to neuronal activity and detectable using noninvasive imaging methods.

In some embodiments, methods and compositions provided herein improve sensitivity of molecular neuroimaging by applying a strategy involving synthetic biology—the engineering of biological systems themselves, rather than introduction of conventional chemical or biomolecular probes. In some embodiments, methods provided herein involve reengineering functional hyperemia, a fast and potent physiological reflex to generic neural activity that is the basis of current hemodynamic functional imaging techniques.

### Neuroimaging Using Synthetic Biology: Reengineering Functional Hyperemia.

In some embodiments, functional hyperemia relates to increases in blood flow triggered by elevations in neural activity levels during stimuli or behavioral tasks. In some embodiments, blood flow increases in turn lead to changes in cerebral blood volume and oxy/deoxyhemoglobin balance that are part of a hemodynamic response to increased neural activity.

In some embodiments, the spatial and temporal resolution of reengineered functional hyperemia is relatively high. In some embodiments, normal hemodynamic responses to neuronal activity have a rise time of several seconds, but onset of hemodynamic signals is much faster and may reflect highly localized components of neural activity. In some embodiments, for comparison, response time of a hypothetical molecular sensor to 100 nM of an analyte under pseudo-first order conditions would generally be on the order of 10 s (assuming kₒₙ=10⁶ M⁻¹s⁻¹), so a rise time of the hemodynamic response is comparable. In some embodiments, although point spread functions for BOLD fMRI responses have been reported to be on the order of 1 mm, structural units that mediate neurovascular coupling operate at a microscopic level, with vascular smooth muscle and even individual capillaries (<10 μm diameter) under neural control. In some embodiments, artificial hyperemic mechanisms engineered to operate primarily on these units are capable of achieving spatial resolution considerably better than conventional functional imaging. In some embodiments, functional imaging weighted towards a signal from smaller blood vessels can be performed using MRI and may be both faster and more spatially precise than other forms of fMRI. In some embodiments, by using a specific mechanism that ties hemodynamic signals to molecular parameters of neural activity, endogenous hemodynamics provides a local and molecularly-specific signal.

In some embodiments, biochemical events that lead to normal functional hyperemia involve nitric oxide synthase (NOS) and cyclooxygase (COX)-mediated pathways as intermediaries. In some embodiments, reengineering functional hyperemia to permit detection of analytes and neuronal signaling events involves inhibiting endogenous hyperemic responses and then adding back to the system an artificial hyperemic response to a molecular or cellular target of interest.

In some embodiments, substantial or complete suppression of functional hyperemia, with minimal perturbation to neural activity, has been achieved using injectable BBB-permeable NOS inhibitors; inhibition has also been achieved with COX, cytochrome P450, and adenosine (A2A) receptor blockers. In some embodiments, NOS or combined inhibition is transient, lasting only for the duration of functional imaging. In some embodiments, transient inhibition is not particularly advantageous where an engineered hyperemic response overwhelms background signal or where measurements are conducted under conditions where background hemodynamic activity averages away. Thus, in some embodiments, methods are provided that involve monitoring hemodynamic parameters for which the signal-to-background ratio of artificial hyperemia is maximized.

In some embodiments, numerous biochemical pathways can be manipulated in a precise manner to bring about artificial hyperemic responses in the presence of inhibited endogenous hemodynamics. In some embodiments, several of these pathways are sensitive to nanomolar-level concentrations of biomolecular modulators, including some that do not normally participate in functional hyperemia, indicating that certain neurovascular responses can act as amplifiers for submicromolar molecular signals. In some embodiments, amplification, made possible because of the intrinsic magnetic and optical properties of blood, represents a strength of certain imaging method disclosed herein. In some embodiments, a further strength relates to the induction of artificial functional hyperemia using a variety of genetic and nongenetically-controlled strategies.

### Engineering Artificial Hyperemia.

In some embodiments, protein engineering approaches are used to develop a variant of neuronal nitric oxide synthase (nNOS) that resists inhibition by selective nNOS blockers. nNOS is implicated in normal functional hyperemia; blockade of nNOS by the selective inhibitors ARL-17477 or L-nitroarginine (L-NNA) abolishes or sharply reduces fMRI-detectable responses to stimulation.

In some embodiments, a nNOS isoform utilizes calcium-bound calmodulin (Ca₄CaM) as a cofactor and thus catalyzes nitric oxide (NO) formation in a highly neural activity-dependent fashion. Another NOS variant, the so-called inducible NOS (iNOS), differs from nNOS in its sensitivity to several inhibitors, and in its independence from calcium ion concentrations (CaM is constitutively bound). In some embodiments, a mutant enzyme that contains the iNOS catalytic domain but the nNOS regulatory and reductase domains retains calcium dependence typical of nNOS. In some embodiments, since inhibitor sensitivity is generally on the structure of the catalytic domain, this nNOS-iNOS chimera is an enzyme with the ability to restore genetically targetable NOS-dependent functional hyperemia responses in the presence of a selective nNOS inhibitor.

In some embodiments, a small panel of nNOS-iNOS chimeras are constructed and tested for their sensitivity to Ca4CaM and to selective nNOS inhibitors. In some embodiments, further protein engineering, and/or synthetic modification to the inhibitors, may be performed to obtain desired specificity profiles. In some embodiments, the gene for inhibitor-resistant nNOS is transduced into neuronal cells using viral vectors to test for restoration of functional hyperemia in the presence of nNOS inhibition in vivo. In some embodiments, unlike experiments involving genetically-encoded MRI contrast agents, particularly high levels of variant nNOS expression are not utilized; moreover, further contrast agents or metal ions are not administered. In some embodiments, NO production is on a par with endogenous levels, and disruption to neuronal processing itself is also minimal.

In some embodiments, the variant nNOS construct is targeted to genetically-defined neuronal populations. In some embodiments, this may be performed either using recombinase-activated constructs or cell type-specific promoters, and may also be performed in conjunction with viral vectors to transduce a subject, e.g., a mouse or rat. In some embodiments, for whole-brain transfection, BBB-permeable viral vectors or an ultrasound-based technique may be used for transient BBB disruption.

In some embodiments, to produce artificial hyperemia without the need for genetic manipulations, variants of a vasoactive molecule called calcitonin gene-related peptide (CGRP) are provided. CGRP is a potent peptidic vasodilator, with an 50%-effective concentration (EC50) below 10 nM for vasodilation of intracerebral arterioles. In some embodiments, the dominant α isoform of CGRP is a 37 amino acid polypeptide that acts via a family of G-protein coupled receptors on vascular smooth muscle cells. In some embodiments, CGRP is naturally released from trigeminal ganglia. In some embodiments, normal CGRP levels in the cerebrospinal fluid are only in the picomolar range. In some embodiments, injected CGRP produces vasodilation and MRI-detectable responses at concentrations 1000 times lower than those utilized for the neurotransmitter-sensitive MRI contrast agents. In some embodiments, to perform target-specific functional imaging with CGRP variants, protein engineering is used to generate ligand-sensitive variants of CGRP. In some embodiments, CGRP is conjugated to a tethered neurotransmitter analog and to a neurotransmitter binding protein in such a way that intramolecular binding between the tethered analog and binding domain reversibly disrupts CGRP's ability to induce vasodilation. In some embodiments, in the presence of the neurotransmitter target itself, intramolecular binding is competitively suppressed, restoring CGRP activity. In some embodiments, because of the sensitivity of brain vasculature to CGRP, only nanomolar concentrations of CGRP-based agents are delivered, meaning that the barrier against trans-BBB permeation is significantly lower than that for a conventional MRI contrast agent or optical dyes.

In some embodiments, brain delivery of agents on this scale may be accomplished using so-called molecular trojan horses, such as transferrin or insulin, which are naturally transported into the brain and have been shown to deliver bioactive amounts of conjugated biomolecular cargo. In some embodiments, CGRP is used as a genetically encoded reporter to report neuropeptide release events.

In some embodiments, methods and compositions provided herein are useful for sensitive detection of neurotransmitters and neuropeptides in vivo. In some embodiments, this detection is accomplished by expressing engineered nNOS, secretable CGRP analogs, or other molecules in genetically targeted cells. In some embodiments, information about spatial and temporal characteristics of chemical signaling events is obtained on a whole brain level. In some embodiments, methods and compositions provided herein enable the assessment of long-range functional connectivity and resting state activity in the brain. In some embodiments, by associating artificial hyperemic responses directly to specific excitatory and inhibitory neural cell types, as well as to neural populations with anatomically distinct projection patterns, it is possible to functionally dissect mechanisms of functional connectivity and determine how information flow is mediated by the different cell groups.

In some embodiments, methods and compositions provided herein enable robust molecule- or cell-specific functional imaging with methods using noninvasive techniques including DOT or photoacoustic tomography. In some embodiments, methods and compositions provided herein are useful for assessing activity on a time scale of seconds. However, in some embodiments, slower changes in gene expression or synaptic plasticity are sensitively detected.

Reagents and Materials

In some embodiments, nucleic acids and viral vectors encoding bioactive (e.g., vasoactive) agents or engineered derivatives are provided. In some embodiments, nucleic acids and viral vectors encoding CGRP or engineered derivatives are provided. In some embodiments, fusion proteins are provided that comprise CGRP fused to protein domains which inhibit CGRP activity. In some embodiments, fusion proteins are provided that comprise CGRP fused to analyte binding domains and analyte analogs, such that the entire fusion is not vasoactive in isolation but can be rendered vasoactive by free analyte which disrupts the intramolecular interaction between binding domain and analyte analog.

In some embodiments, fusion proteins are provided that comprise CGRP fused to an inactivating domain which can be become active upon enzymatic modification (for example, upon proteolytic cleavage). In some embodiments of the fused proteins, the CGRP portion is a mutant (e.g., a mutant with altered target affinity).

Methods of using reagents for functional brain imaging via the hemodynamic response include the use of natural CGRP or of engineered derivatives. The reagents may be injected as peptides or delivered in the form of a nucleic acid, for example using viral vectors, for in situ expression of the CGRP constructs. Expression of CGRP constructs from promoters which are specific to certain cell types or cell states, and can report such cell states. In some embodiments, CGRP derivatives are provided that are conditionally active, e.g., have a biologically inactive state but can become activated by binding or by catalytic action of an analyte of interest. In some embodiments, CGRP derivatives are provided whose secretion is controlled by a signal of interest. In some embodiments, optimized and validated protocols are provided for (i) engineering CGRP derivatives and other reagents and for (ii) performing in vivo imaging using such materials.

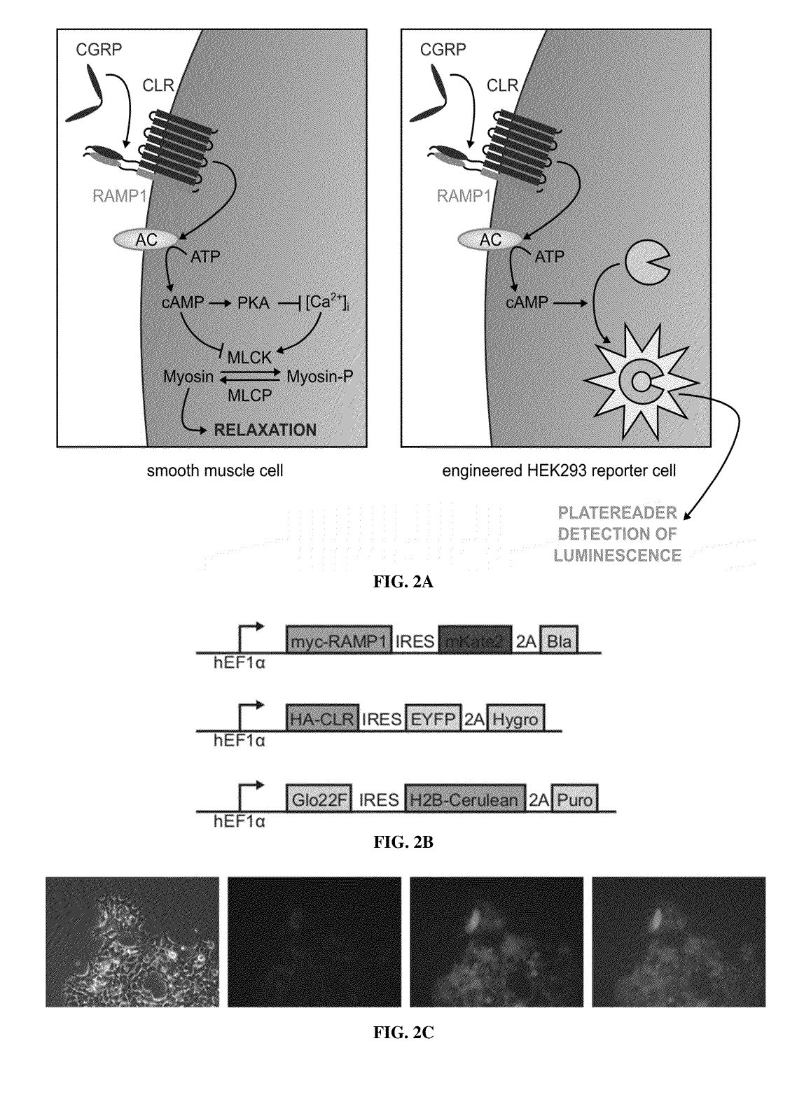

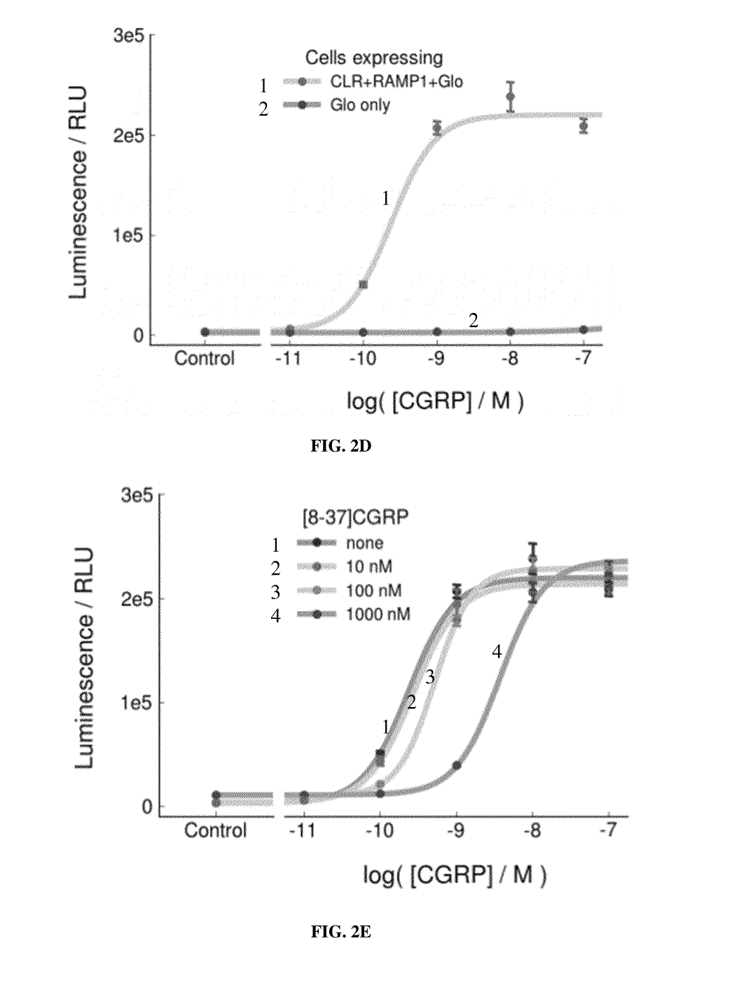

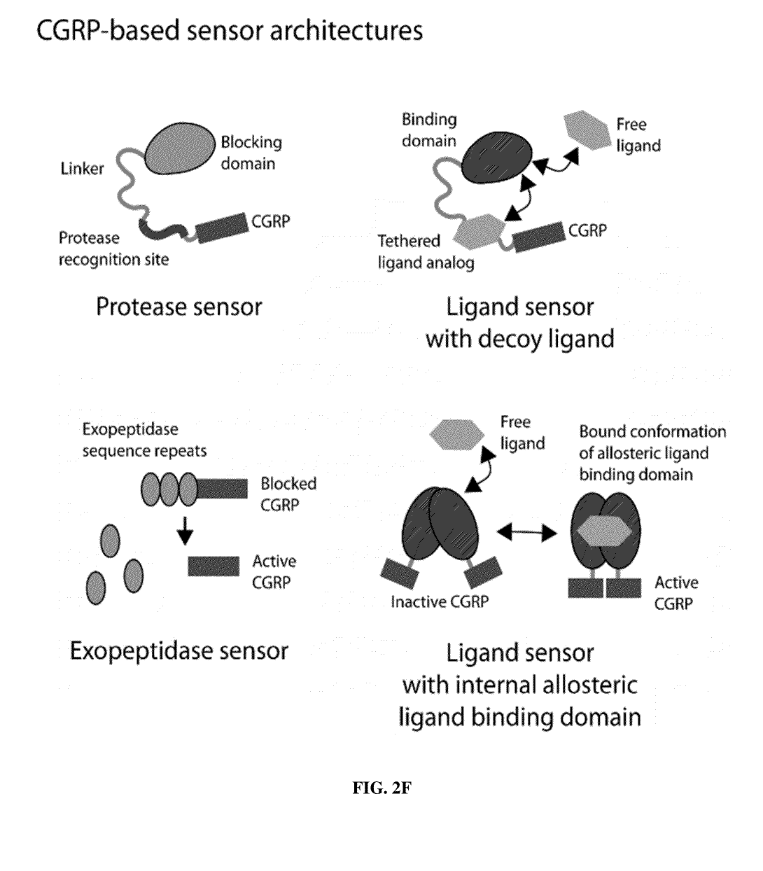

In some embodiments, transgenic expression constructs for CGRP or engineered derivatives are provided that have a coding region for CGRP or an engineered derivative placed under the regulatory control of promoters of interest to achieve specific expression depending on tissue or cell type or on gene regulatory events. In some embodiments, CGRP or engineered derivatives may be transgenically expressed, or injected, in brain structures of interest and perform their action only there, simplifying the mapping of measured signals onto a brain atlas. Ligand-sensitive CGRP derivatives have been designed for the detection of specific molecular species such as neurotransmitters (e.g., as depicted in FIG. 2).

Further molecules are provided for detection of catalytic activity, such as that of matrix metalloproteases involved in tumor metastasis or proteases involved in apoptosis.

In some embodiments, an engineered chimera is provided that has a catalytic domain of inducible NOS (NOS) fused to a regulatory domain of neuronal NOS (nNOS). The chimera exhibits dependence on calcium-bound calmodulin (Ca₄CaM) as conferred by the nNOS regulatory domain, and independence of certain synthetic, BBB-permeable inhibitors with specificity for the nNOS catalytic domain such as 7-nitroindazole (7-NI). Furthermore, calcium detection by engineered NOS may be used as a tool for measuring brain activity.

In some embodiments, compositions are provided that comprise a compound (e.g., a protein, engineered protein or chimera) and a carrier, e.g., a pharmaceutically acceptable or physiologically acceptable carrier.

The term “carrier” denotes an organic or inorganic ingredient, natural or synthetic, with which the active ingredient is combined to facilitate the application. As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

Compositions of the invention can be administered by any conventional route, including injection or by gradual infusion over time. The administration may, for example, be oral, intravenous, intrathecal, intraneural, intracerebral, intraparenchymal, epidural, intratumoral, intraperitoneal, intramuscular, intracavity, subcutaneous, or transdermal. The composition can be administered in an effective amounts. An “effective amount” is an amount of a compound or composition that alone, or together with further doses, produces the desired response, e.g., a hemodynamic response, either directly or indirectly.

In certain aspects of the invention, kits are provided, comprising a container housing a compound or composition. In some embodiments, individual components of a composition may be provided in one container. Alternatively, it may be desirable to provide the components of the composition separately in two or more containers, e.g., one container for a compound (e.g., a vasoactive agent), and at least another for a carrier. The kit may be packaged in a number of different configurations such as one or more containers in a single box or package. The different components can be combined, e.g., according to instructions provided with the kit. The components can be combined according to a method described herein, e.g., to prepare and administer a compound or composition. The kit can also include a delivery device.

The present invention is further illustrated by the following Examples, which in no way should be construed as further limiting.

### EXAMPLES

### Example 1

### Molecular Functional Imaging Using Engineered Neuropeptides

Calcitonin gene-related peptide (CGRP) is a 37 amino acid, vasoactive neuropeptide which is secreted by neurons and effects dilation of the microvasoulature by binding to a heterodimeric, G-protein coupled receptor on the surface of endothelial cells. CGRP is a potent peptidic vasodilator, with half-maximal effect at a concentration below 10 nM.

CGRP receptors are expressed inside the brain and in the presence of an intact BBB, intravenously injected CGRP has no detectable vasodilatory effect in the brain vasculature. Disruption of the BBB by traumatic injury, carcinogenesis, or inflammation renders it penetrable by CGRP. Thus, BOLD MRI following systemic CGRP injection may be used to detect BBB disruption.

Other vasoactive peptides used include engineered adrenomedullin (ADM) and especially engineered Maxadilan (Max), a vasoactive peptide from the salivary glands of sandflies which causes vasodilation in mammals much like CGRP (see, e.g., U.S. Pat. No. 5,480,864). In some embodiments, Maxadilan is advantageous because it has a relatively high potency (e.g., greater than CGRP by a factor of 100). In some embodiments, the EC50 for cAMP elevation by Maxadilan is in the picomolar range. In some embodiments, Maxadilan is advantageous because it is comprised of over 60 amino acids, many of which can be altered with minor impact on potency and activity. In some embodiments, this feature permits a ligand-binding allosteric domain to be inserted at one of the ends or in the middle of the molecule such that a fusion polypeptide has maxadilan-like activity in the presence but not the absence of a ligand of interest. In some embodiments, Maxadilan is advantageous because of the wide and relatively even expression in the brain of the PAC1 receptor, which is activated by maxadilan.

In some embodiments, CGRP receptor is advantageous because it triggers minimal physiological responses apart from vasodilation. Activation of the PAC1 receptor may trigger pleiotropic effects, which will involve assessment for each particular analyte and imaging test as to whether they are acceptable for the purposes of the imaging objective.

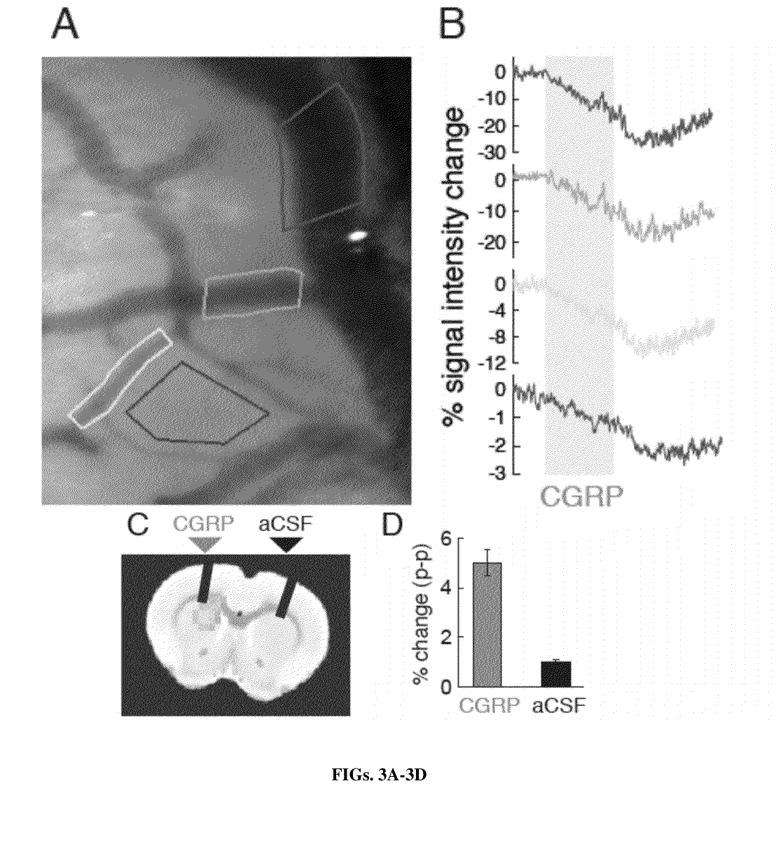

Artificial hyperemia was induced by CGRP injection in the brains of live animals. Results are presented in FIG. 3. FIG. 3A shows optical image of exposed cortex highlighting vascular regions of interest (ROIs). FIG. 3B shows percent signal changes over time for ROIs coded in panel of FIG. 3A; 10 nM CGRP was infused during the shaded period. FIG. 3C shows an MRI map showing areas of significant signal change (colored voxels) due to 100 nM CGRP infusion through a cannula in the left hemisphere. Control infusion of cerebrospinal fluid on the right side produced no significant changes; group data (n=4) are quantified in FIG. 3D.

### Example 2

### Molecular Functional Imaging Using Engineered NOS

Nitric oxide synthase (NOS) is an intermediary in the hyperemic response, and exists in several isoforms which differ in their responsiveness to regulatory factors.

In this example, a strategy is provided for molecular functional imaging of the brain using engineered NOS. An engineered chimera is produced that has a catalytic domain of inducible NOS (NOS) and of the regulatory domain of neuronal NOS (nNOS). The chimera exhibits dependence on calcium-bound calmodulin (Ca₄CaM) as conferred by the nNOS regulatory domain, and independence of certain synthetic, BBB-permeable inhibitors with specificity for the nNOS catalytic domain such as 7-nitroindazole (7-NI).

Using the chimeras, neural activity and calcium release is related to NOS activity by (i) inhibition of endogenous nNOS activity using systemic administration of 7-NI and (ii) calcium-dependent activation of the nNOS-iNOS chimeras. Imaging of the resulting hemodynamic response is accomplished by optical imaging and/or BOLD MRI.

The engineered chimeras are delivered to tissues using any appropriate method. For example, the chimeras are directly or indirectly administered to the tissue. Alternatively, transgenes engineered to express the chimera are delivered to a tissue (e.g., brain tissue) of a live animal (e.g., a mouse or rat) using viral vectors. In some instances, the viral vectors are engineered to target specific cell types of interest in the animal.

This strategy is useful (e.g., in a neurophysiological research context) for the deconvolution of calcium signaling (after stimulation, or during behavioral exercises) by genetically defined cell type, in addition to spatiotemporal resolution of the activity. For example, by targeting the chimeras to only dopaminergic neurons and inhibiting endogenous nNOS activity, calcium release in this cell type can be recorded by BOLD MRI.

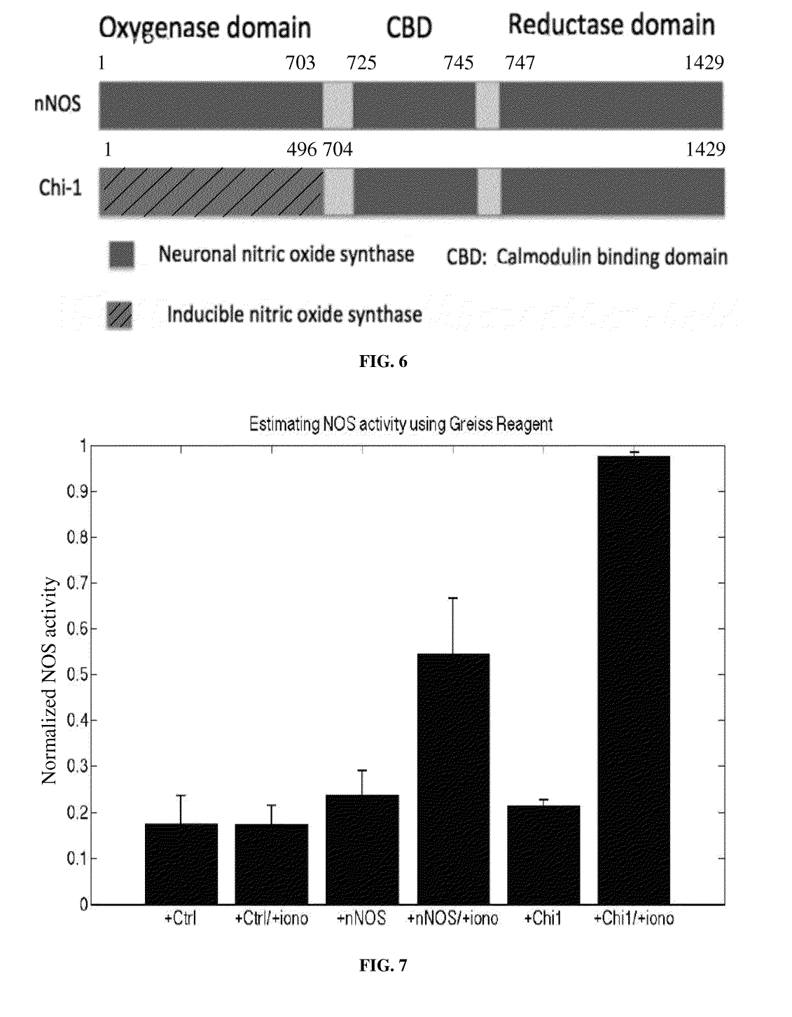

FIG. 6 shows engineered nitric oxide synthases (NOS). To artificially trigger hemodynamic response using NOS, a chimeric NOS, (Chi-1) was expressed that consists of the oxygenase domain of the human iNOS and the calmodulin-binding domain and the reductase domain of the rat nNOS.

To assess the extent to which Chi-1 had calcium inducible NOS activity, rat nNOS and Chi-1 were expressed in HEK 293 cells using transient transfection (FIG. 7). 48 hours post transfection, 1 mM LArginine and 5 μM A23187 (a calcium ionophore) were added to induce activity. Nitrite formation was measured 8 hours post induction using Greiss Reagent.

These constructs may be used in the presence of isoform-selective NOS inhibitors. In some embodiments, engineered NOS will be enzymatically active in presence of an nNOS selective inhibitor (ARL-17477) but not in the presence of an iNOS selective inhibitor (1400-W). In some embodiments, these variants may be used for in vivo applications.

### Example 3

### A CGRP-Based Molecular Sensor

A CGRP-based molecular sensor for neurotransmitter concentrations is constructed as a fusion protein comprising a neurotransmitter analog, CGRP, and a binding domain such that the interaction between the binding domain and the analog disrupts CGRP function. In the presence of the neurotransmitter of interest, the free neurotransmitter is bound by the binding domain instead and CGRP function is (reversibly) restored. Thus, vasodilation is induced in response to the neurotransmitter with spatial and temporal specificity and recorded by optical or magnetic resonance imaging.

CGRP reporter cells were created by lentiviral transduction of HEK293FT cells with genes encoding the heterodimeric CGRP receptor, comprised of human CALCRL and RAMP1, and a luminescent reporter. HEK293FT cell lines carrying the following lentiviral constructs were used:

(SEQ ID NO: 16)
pEXPR-T7-cfSGFP2-Linker-Cleavage-Site-CGRP-Gly
(SEQ ID NO: 17)
pLV-hEF1a-GLo22F-IRES-H2B-Cerulean-2A-puro
(SEQ ID NO: 18)
pLV-hEF1a-HA-CALCRL-IRES-EYFP-2A-Hygro
(SEQ ID NO: 19)
pLV-hEF1a-myc-RAMP1-IRES-mKate-2A-bla

Glo22F is a gene, offered by Promega Corp., encoding an engineered luciferase whose activity is allosterically modulated by cyclic AMP (Fan et al. “Novel Genetically Encoded Biosensors Using Firefly Luciferase”. ACS Chem. Biol., 2008, 3 (6), pp 346-351 DOI: 10.1021/cb8000414). This luminescent cAMP reporter system offers relatively fast readouts from live cells and strong signal-to-noise ratio.

CGRP-based molecular sensors may be produced by recombinant expression in E. coli followed by extraction, purification, and refolding. In this regard, fusion proteins of the general structure have been developed: StrepII-GFP-(linker)-(protease site)-CGRP-Gly, where StrepII is an affinity tag for purification, GFP is one example of a large globular blocking domain, and CGRP-Gly is human alpha CGRP extended with a single glycine residue in lieu of C-terminal amidation. The StrepII affinity tag can be found from amino acids 1 through 13, the cfSGFP2 blocking domain (PLoS One. 2012; 7 (5):e37551. Development of cysteine-free fluorescent proteins for the oxidative environment. Suzuki T, Arai S, Takeuchi M, Sakurai C, Ebana H, Higashi T, Hashimoto H, Hatsuzawa K, Wada I.) can be found from amino acids 14 through 251, the linker and protease site can be found from amino acids 252 to 252+n where n can be any number of amino acids (e.g., 1 to 10, 1 to 50, 1 to 100 amino acids). haCGRP-Gly: Human alpha calcitonin gene-related peptide extended with one C-terminal glycine residue spans from amino acid 252+n+1 to the end of the peptide sequence. The general class of polypeptides that function as CGRP-based protease sensors have the following general amino acid sequence:

(SEQ ID NO: 14)
MSAWSHPQFEKGAVSKGEELFTGVVPILVELDGDVNGHKFSVSGEGE
GDATYGKLTLKFISTTGKLPVPWPTLVTTLTYGVQMFARYPDHMKQH
DFFKSAMPEGYVQERTIFFKDDGNYKTRAEVKFEGDTLVNRIELKGI
DFKEDGNILGHKLEYNYNSHNVYITADKQKNGIKANFKIRHNIEDGG
VQLADHYQQNTPIGDGPVLLPDNHYLSTQSKLSKDPNEKRDHMVLLE
FVTAAGITLGMDELYK-(Linker)-(Protease site)-
(SEQ ID NO: 15)
ACDTATCVTHRLAGLLSRSGGVVKNNFVPTNVGSKAFG.

In some embodiments the linker and protease site can have the following amino acid sequences or combinations of amino acid sequences which comprise a Caspase-3 sensor with a GGSG (SEQ ID NO: 13) linker and DEVD (SEQ ID NO: 9) protease site, a TEV protease sensor with a GGSG (SEQ ID NO: 13) linker and ENLYFQG (SEQ ID NO: 11) protease site, an Enterokinase sensor with a GGSG (SEQ ID NO: 13) linker and a DDDDK (SEQ ID NO: 12) protease site, a MMP-9 sensor with a GGSG (SEQ ID NO: 13) linker and GPLGIAG (SEQ ID NO: 8) protease site, or a Factor Xa sensor with a GGSG (SEQ ID NO: 13) linker and a IEGR (SEQ ID NO: 10) protease site.

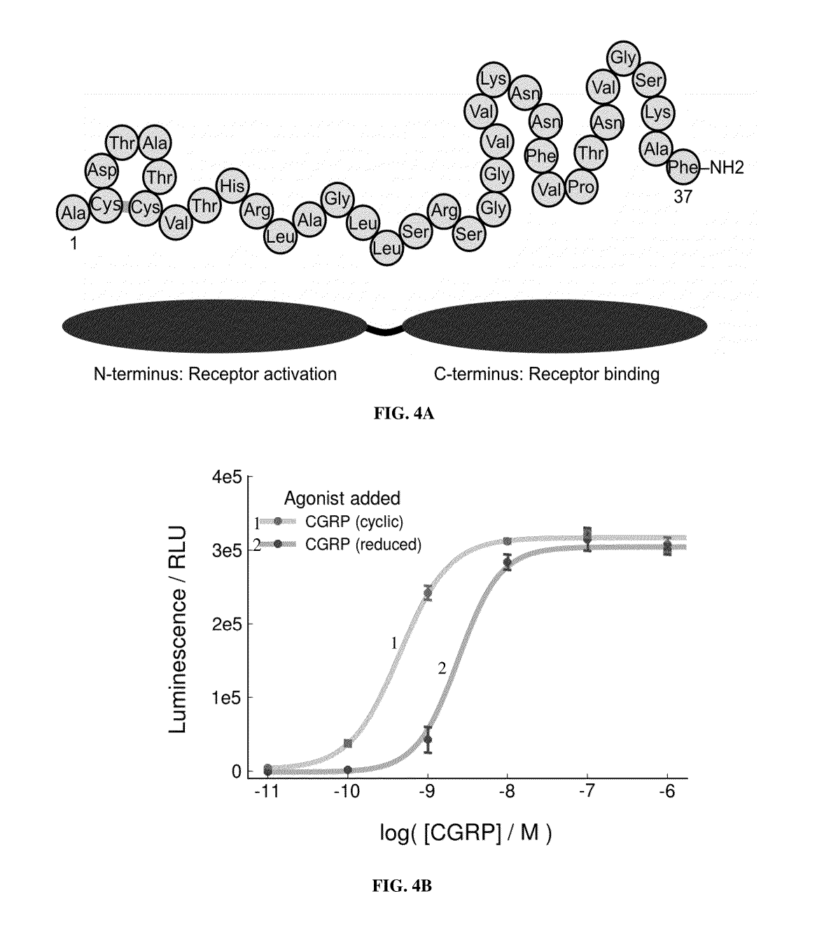

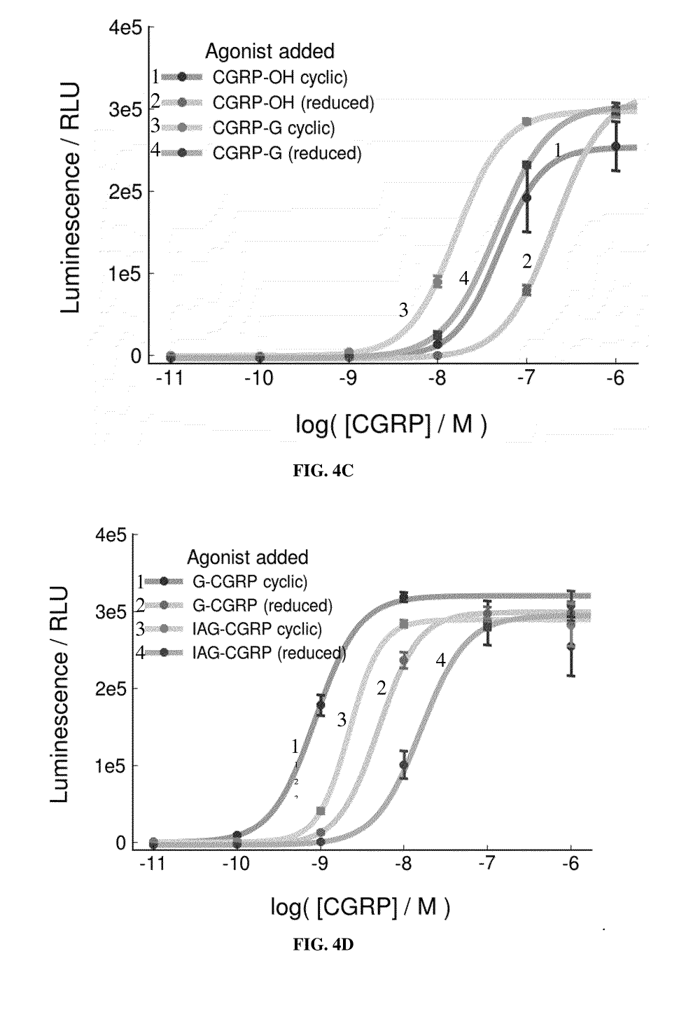

It has been shown that synthetic CGRP-Gly peptide fully activates the CGRP receptor in cell culture with nanomolar potency. Fusions of the structure described above, with protease sites specifically enabling cleavage by, for example, caspase-3; TEV protease; and enterokinase were unable to activate the CGRP receptor in the cell-based bioassay even at high micromolar concentrations when uncleaved; and able to fully activate the CGRP receptor after being cleaved by their respective protease. See FIGS. 4A-4D and 5A-5D.

### Example 4

### Assessment of Traumatic Brain Injury (TBI) Overview

Acute neurotrauma resulting from blast exposure or impact injury is accompanied by inertial and shearing forces that mechanically injure nerve cells, nerve fiber tracts, and small blood vessels in the brain. These acute injuries lead to neuronal dysfunction, axonal conduction defects, blood-brain barrier (BBB) disruption, and focal neuroinflammation that clinically manifest as neurobehavioral and cognitive deficits. These clinical symptoms often resolve over time. However, in susceptible individuals, acute TBI may trigger pathogenic cascades leading to chronic neurological sequelae, including chronic traumatic encephalopathy (CTE). Perivascular tau accumulation and chronic neuroinflammation characterize the earliest stages of CTE neuropathology, thus implicating microvascular pathology as a presumptive factor linking acute TBI to later development of CTE.

Understanding the pathogenesis of TBI and CTE is important for development of diagnostics and therapies for these disorders. This example relates to a noninvasive molecular imaging technology that elicits hemodynamic signal changes near TBI-associated vascular lesions. Results may be compared with complementary assessments of focal BBB disruption following injury. The relationship between BBB disruption and other markers of TBI- and CTE-related neuropathology that colocalize with the neuroinflammatory damage induced by the acute injury, making use of a highly sensitive post mortem metallomic imaging technique, are correlated with results from in vivo molecular imaging. The methods provide an option for multimodal clinical or point-of-care diagnosis of TBI, in addition to helping to characterize a fundamental aspect of neurotraumatic injury.

### Perivascular Microstructural Pathology in Traumatic Brain Injuries.

Exposure to blast from conventional and improvised explosive devices (IEDs) affects combatants and civilians in conflict regions around the world. Blast-exposed individuals are subject to traumatic brain injury (TBI) with debilitating neuropsychiatric symptoms, cognitive deficits, and neurological sequelae that resemble the clinical signs and symptoms of head-injured athletes diagnosed with chronic traumatic encephalopathy (CTE). CTE is a progressive tau protein-linked neurodegenerative disease associated with repetitive concussive and subconcussive head injury. Neuropathological hallmarks of CTE include cortical foci of perivascular tau pathology, disseminated microgliosis, astrocytosis, and axonal degeneration. Clinical symptoms of CTE include depression, irritability, distractability, executive dysfunction, memory loss, and in advanced cases, cognitive deficits and dementia. Blast exposure is a known precipitant of TBI in humans and animals.

Neuropathological abnormalities associated with traumatic injuries indicate a pathogenic mechanism involving blood-brain barrier (BBB) disruption induced by the shearing forces associated with the inciting trauma. Disruption of cerebrovascular integrity leads to focal microhemorrhage, parenchymal hemolysis and iron accumulation, local reactive oxygen species generation, and increased oxidative stress. A secondary neuroinflammatory cascade may occur which leads to perivascular tau accumulation and cerebrovasculature abnormalities that define CTE neuropathology. In this context, CTE neuropathology may follow a common pathogenic pathway that is independent of the particular context of the inciting brain injury. Perivascular CTE neuropathology in football players with repetitive head injury is similar or identical to that observed in combat soldiers with TBI caused by repetitive IED blast exposure.

### Vascular Pathology as a Basis for Diagnosing TBI and CTE.

Shared cerebrovascular abnormalities and perivascular inflammatory markers in TBI and CTE indicates an alternative and powerful avenue for diagnosis and mechanistic analysis of these conditions. For diagnostic purposes in particular, identification of neurovascular biomarkers of traumatic injury could complement techniques such DTI, which aim to detect subtle abnormalities in the microarchitecture of neural tissue itself.

Methods are provided herein to quantify the extent of vascular injury and BBB disruption. Furthermore, methods are provided for detection of BBB disruption, and to associate experimental measures with other indications of neuropathology, initially in animal models of mild TBI.

Provided herein are sensitive molecular tools for characterizing BBB disruption that are well suited to detecting perivascular trauma in mild TBI and have a positive impact on diagnosis. Thus, a class of imaging agents is provided herein that acts at nanomolar concentrations to elicit artificial hemodynamic responses detectable by MRI and other imaging modalities. Because these probes are effective in the brain parenchyma, but not in the vasculature, they provide a highly sensitive indication of BBB disruption.

A validated rodent model of blast injury provides a context in which to evaluate the probes as potential diagnostic agents for TBI and CTE.

### Diagnostic Strategy for Sensitive In Vivo Detection of Vascular Lesions in Mild TBI.

A diagnostic strategy is provided for sensitive in vivo detection of vascular lesions in mild TBI. A class of vasoactive imaging agents is provided to reveal subtle BBB disruptions associated with mild TBI. Imaging agents are applied in a rodent model of blast injury and detection is performed using magnetic resonance imaging (MRI). Results using the vasoactive agents are compared with those obtained using conventional MRI contrast agents, which constitute the clinical standard for assessment of BBB disruption, but appear to offer limited sensitivity for detection of vascular damage in mild TBI.

Provided is an approach to molecular imaging that is particularly suitable for detection of mild BBB disruption in TBI. In this approach, image signals are induced by a vasoactive molecule, rather than by a conventional imaging agent. A potent vasodilating biomolecule, the calcitonin gene related peptide (CGRP) is utilized in this example. But other vasoactive probes, including small molecules as well as additional polypeptides, may be used.

Using intracranial injection, detectable contrast in MRI is induced by nanomolar-scale concentrations of CGRP, 1000-fold lower than concentrations associated with conventional MRI contrast agents. The contrast is induced when CGRP is in the brain tissue; no contrast is induced by intravascular administration. This is due to the fact that CGRP receptors are found only in the brain parenchyma, and indicates that leakage of CGRP from the vascular lumen to the parenchyma would induce contrast potentially specific to areas of compromised BBB function. In some embodiments, this molecular imaging approach is referred to as “engineered hemodynamic” contrast. In some embodiments, the approach is useful for ultrasensitive imaging both in TBI and in other diseases associated with BBB dysfunction.

The approach is evaluated using MRI, which provides well known sensitivity to hemodynamic effects. Engineered hemodynamic contrast is suitable for detection by additional methods however, including diffuse optical imaging and photoacoustic tomography methods and others that provide lower resolution images than MRI but are easy to implement at points of care.

Results show that intracranial injection of CGRP elicits MRI contrast, with dynamic MRI signal changes on the order of several percent in response to infusion of 0.1 μM peptide solution. In parallel, optical imaging changes in due to infusion of CGRP to exposed rat cortex in a cranial window preparation were detected. In experiments where CGRP was peripherally injected into tail vein, no detectable signal changes were observed. CGRP delivered intravascularly to animals with perturbed BBB results in analogous signal changes.

Efficacy for imaging BBB leakage in TBI is tested using rats subjected to pressure shocks in a blast tube shown to simulate IED exposure in humans. This fully validated model enables sublethal blasts with quantitatively controlled pressure waveforms to be reproducibly administered with 100% survival of subjects. Following blast treatment, animals are injected with CGRP at doses shown to produce artificial hemodynamic responses. Intravenous CGRP injection is performed using an MRI compatible syringe pump and time-resolve image series are acquired using T2 and T2*-weighted MRI pulse sequences implemented on a Bruker 7 T 20 cm bore scanner. Data is transformed and processed in Matlab, using analysis routines already established for evaluation of hemodynamic responses.

When responses are weak or unobserved, both the CGRP dose and blast amplitude are adjusted to determine a threshold for detection. Animals are sacrificed after imaging to evaluate tissue pathology and quantify BBB disruption, and to detect CGRP directly in the post mortem brain.

Positive control experiments are performed using a Gd-DTPA infusion method, and sensitivity of CGRP vs. Gd-based detection is quantitatively compared, with CGRP being effective at far lower doses. The pharmacological dependence of engineered hemodynamic responses is probed by examining CGRP-mediated contrast in the absence of CGRP antagonists or probes that interfere with alternative hemodynamic mechanisms, such as blood oxygen level dependent (BOLD) contrast in functional MRI.

### Assessment of BBB Integrity and Focal Disruption Using Metallomic Imaging.

Furthermore, BBB integrity and focal disruption are assessed using metallomic imaging. The extent of BBB disruption post mortem in blast model animals is assessed using an extremely sensitive metallomic imaging approach that detects localized extravasation of erythrocytes and metallic tracers associated with vascular lesions in mild TBI. Results are compared with the molecular imaging data, to evaluate the sensitivity of the noninvasive imaging approach. Data are also compared with other histological markers associated with TBI or CTE.

A complement to the exploratory diagnostic strategy is the unambiguous characterization and quantification of TBI-related vascular brain damage by post mortem analytical techniques. Animals subjected to blast treatment are examined by metallomic imaging and histopathological analysis for comparison of results to molecular imaging data.

Assessment of BBB integrity and focal disruption are performed using laser ablation inductively-coupled plasma mass spectrometry (LA-ICP-MS) implemented to assess BBB integrity and focal disruption. BBB disruption results in accumulation of hemolytic iron, cytokines, and other neuroinflammatory stimuli in the surrounding brain tissue and extracellular fluid. Because these processes involve redistribution of specific metal ions (chiefly iron), the effects are directly measured by examining elemental distribution in brain sections obtained from blast model animals. High-resolution metallomic analytical capabilities that utilize a magnetic sector field LA-ICP-MS instrument (Element-XR, Thermo Scientific, Bremen, Germany) provides definitive, interference-free mass identification (<0.005 amu), broad linear dynamic range (LDR>1012), and ultra-trace sensitivity (ppt/ppq) for elements and isotopes across the periodic table. Hyphenation with femtosecond infrared laser ablation cryogenic sampling enables multi-channel ultra-trace metallomic mapping with unparalleled analytical sensitivity at single-cell spatial resolution. HR-MIMS provides an approach to anatomically localize and analytically quantitate blood-brain barrier dysfunction that is not possible using conventional techniques (e.g. Evans Blue).

High sensitivity metallomic imaging is complemented by a battery of more conventional histopathological analyses. Temporal and regional patterns of microvascular disruption are characterized using three semiquantitative techniques: Evans Blue (EB) extravasation by quantitative fluorescence, brain edema assessment and IgG immunohistochemistry. Typically, BBB compromise produces site-specific extravasation resulting in the leakage of microvascular contents into the brain parenchyma. Methods for quantification of BBB compromise are used. Briefly, EB (4 ml/kg in 2% saline) is injected intravenously 30 minutes before BNT. Sub-quantitative results are analyzed by fluorescence (excitation, 620 nm; emission, 680 nm) and expressed as fluorescence intensity per gram tissue. Correlation analysis assesses focal microvascular disruption as a function of regional neuroinflammation and compared to CTE neuropathology. Edema assessment follows standard protocol using the tissue water weight methods. The spatial statistics of vascular lesions are compared with findings from molecular imaging studies, and spatial correlation of findings from individual animals subjected to MRI and post mortem analysis is performed.

### Example 5

### Laboratory Diagnostic Testing with CGRP-Based Molecular Sensors

Molecular sensors based on CGRP as described in Example 3 and CGRP reporter cells as described in Example 3 can also be used in an in vitro diagnostic assay, in cases where this is appropriate for the analyte of interest and the diagnostic goal. The assay result in this type of application is not an image; the result is a determination of the presence or absence of the analyte, or of the concentration of the analyte, in a biological sample of interest.

In the case of protease sensors, the analyte of interest may be the activity of a protease marker such as Prostate Specific Antigen in a biological sample such as urine and blood as a biomarker for prostate cancer. In the case of ligand sensors, the analyte of interest may be drawn from among the many diagnostic biomarkers currently detected by specific molecular binding events in immunoassays.

Several types of measurement apparatus may be employed to carry out such an assay. For example, in Diagnostic Workflow 1, HEK293FT reporter cells are employed in a microplate-based bioassay as described in Example 3. A biological sample, for example urine, is mixed with a CGRP-based sensor in a suitable detection buffer; the mixture is allowed to react for an appropriate period of time that allows the molecular detection event to take place; it is then serially diluted in buffer; and added to different wells of the multiwall microplate. CGRP receptor activation is then recorded as an optical signal (for example, luminescence) using a microplate reader. Regression analysis of the readout reveals the concentration of the analyte of interest in the sample. Workflow 1 represents a laboratory diagnostic assay.

Workflow 2 represents a point-of-care diagnostic assay using engineered CGRP-based molecular sensors. To implement it, a lateral flow device is employed in which a lyophilized CGRP-based biosensor and suitable reporter cells are deposited in a matrix. The reporter cells for this application are yeast cells functionally expressing a CGRP receptor (for example, as described in Miret J J, et al., Functional expression of heteromeric calcitonin gene-related peptide and adrenomedullin receptors in yeast. J Biol Chem. 2002 Mar. 1; 277(9):6881-7.) and an optical reporter of GPCR activity. A suitable optical readout for a point-of-care diagnostic assay is, for example, expression of a pigment visible to the eye. Live yeast cells are dried, facilitating their use in a point-of-care diagnostic device that may involve extended storage times before use. The diagnostic device further provides buffer components which, when mixed with the intended biological sample (for example, saliva or urine) allows the molecular detection event (for example, proteolytic cleavage of the CGRP-based sensor, or molecular binding to the sensor) to take place. To use the device, a small quantity of the fluid sample is deposited into the device; a defined period of time is allowed to elapse during which sample transport, molecular detection, and reporter cell activation and optical signal generation take place; and the optical readout (such as appearance of a colorful pigment at the location of the reporter cells) is observed for the sample of interest and for appropriate control samples.

CGRP-based molecular sensors offer a potential advantage over other diagnostic methods in that receptor activation on reporter cells provides a specific, sensitive, and highly amplified readout of the detection event. In some embodiments, assays disclosed herein utilize a GPCR ligand (as an analyte-specific sensor.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method for evaluating a tissue in a subject, the method comprising:
producing a hemodynamic response in a tissue of a subject by providing an effective amount of a vasoactive agent to the tissue;
obtaining an image representation of the tissue; and
evaluating the tissue based on a hemodynamic response detected in the image representation.

2. The method of claim 1, wherein the hemodynamic response is produced by providing to the tissue an exogenous vasoactive agent in a submicromolar concentration that is effective for inducing a hemodynamic response in the tissue

3. The method of claim 1, wherein the vasoactive agent is provided to the tissue by administering the vasoactive agent to the subject.

4-21. (canceled)

22. The method of claim 1, wherein the hemodynamic response comprises a change in blood volume in the tissue.

23. The method of claim 1, wherein the hemodynamic response comprises an increase in blood flow in the tissue.

24. The method of claim 1, wherein the hemodynamic response comprises a decrease in blood flow in the tissue.

25. The method of claim 1, wherein the hemodynamic response comprises changes in oxy/deoxyhemoglobin balance in the tissue.

26. The method of claim 1, wherein the tissue is neuronal tissue and wherein the hemodynamic response is associated with changes in neural activity.

27. The method of claim 26 further comprising determining that the hemodynamic response is associated with excitatory neural activity in the tissue.

28. The method of claim 26 further comprising determining that the hemodynamic response is associated with inhibitory activity in the tissue.

29. The method of claim 1, wherein the vasoactive agent is a molecule having the formula X¹-L¹-X²-L²-X³, in which X¹ is a blocking domain and/or ligand binding domain, L¹ and L² are independently linkers or absent, X² is a protease recognition site and/or a ligand or analog thereof, and X³ is a vasoactive molecule.

30-44. (canceled)

45. The method of claim 29, wherein the vasoactive molecule is calcitonin gene-related peptide (CGRP) or an engineered derivative thereof.

46. The method of claim 29, wherein the vasoactive molecule is adrenomedullin (ADM) or an engineered derivative thereof.

47. The method of claim 29, wherein the vasoactive molecule is Maxadilan (Max) or an engineered derivative thereof.

48-73. (canceled)

74. A method for evaluating presence of a molecular analyte in a tissue, the method comprising:
providing to a tissue a vasoactive agent, the activity of which vasoactive agent is modulated by binding to an analyte, wherein modulation of the vasoactive agent results in a hemodynamic response in the tissue;
obtaining an image representation of the tissue; and
evaluating ligand binding by assessing in the image representation presence or absence of a hemodynamic response resulting from modulation of the vasoactive agent in the tissue.

75-101. (canceled)

102. An engineered vasoactive agent having the formula X¹-L¹-X²-L²-X³, in which X¹ is a blocking domain and/or ligand binding domain, L¹ and L² are independently linkers or absent, X² is a protease recognition site and/or a ligand or analog thereof, and X³ is a vasoactive molecule.

103. The engineered vasoactive agent of claim 102, wherein X¹ is a blocking domain, L¹ is a linker, L² is absent, X² is a protease recognition site, and X³ is a vasoactive molecule.

104. The engineered vasoactive agent of claim 102, wherein X¹ is a ligand binding domain, L¹ is a linker, L² is absent, X² is a ligand or analog thereof, and X³ is a vasoactive molecule.

105. A method for evaluating a tissue in a subject, the method comprising:
producing a hemodynamic response in a tissue of a subject by providing an effective amount of a vasoactive agent to the tissue;
detecting spectroscopic signals indicative of the hemodynamic response; and
evaluating the tissue based on the hemodynamic response.

106. The method of claim 105, wherein the spectroscopic signals are detected using optical spectroscopy, magnetic resonance spectroscopy, or localized spectroscopy.
